(19) United States
(12) Patent Application Publication
(10) Pub. No.: US 2024/0408003 A1
(43) Pub. Date: Dec. 12, 2024

(54) COMPOSITIONS, DEVICES AND METHODS FOR THE TREATMENT OF ALCOHOL USE DISORDER

(71) Applicants: Indivior UK Limited, Hull (GB); Aegis Therapeutics, LLC, San Diego, CA (US)

(72) Inventors: Roger CRYSTAL, Santa Monica, CA (US); Arvind AGRAWAL, Santa Monica, CA (US); Edward T. MAGGIO, San Diego, CA (US)

(73) Assignees: Indivior UK Limited, Hull (GB); Aegis Therapeutics, LLC, San Diego, CA (US)

(21) Appl. No.: 18/392,008

(22) Filed: Dec. 21, 2023

Related U.S. Application Data
(63) Continuation of application No. 16/311,944, filed on Dec. 20, 2018, now abandoned.
(60) Provisional application No. 62/419,736, filed on Nov. 9, 2016.
(60) Provisional application No. 62/354,465, filed on Jun. 24, 2016.

(51) Int. Cl. A61K 9/00; A61K 9/08; A61K 31/485; A61K 45/06; A61K 47/02; A61K 47/18; A61K 47/26; A61K 47/36; A61M 11/00; A61M 15/00; A61M 15/06; A61M 15/08; A61P 25/32

(52) U.S. Cl. CPC A61K 9/0043; A61K 9/08; A61K 31/485; A61K 45/06; A61K 47/02; A61K 47/183; A61K 47/186; A61K 47/26; A61M 15/0068; A61M 15/08; A61P 25/32; A61K 47/36; A61M 11/006; A61M 15/06

(57) ABSTRACT

Drug products adapted for nasal delivery, comprising a pre-primed device filled with a pharmaceutical composition comprising naltrexone are provided. Formulations and methods of treating alcohol use disorder and related conditions with the drug products are also provided.

This application is a continuation of U.S. patent application Ser. No. 16/311,944, filed Dec. 20, 2018, which is a U.S. national phase entry under 35 U.S.C. § 371 of International Application No. PCT/US2017/039300, filed Jun. 26, 2017, which claims the benefit of U.S. Provisional Application Nos. 62/354,465, filed Jun. 24, 2016, and 62/419,736, filed Nov. 9, 2016, the disclosures of each of which are hereby incorporated by reference as if written herein in their entirety.

Disclosed herein are methods and compositions for the treatment of alcohol use disorder comprising administering an intranasal formulation of the opioid antagonist naltrexone.

The problematic drinking of alcohol that becomes sufficiently severe is given the medical diagnosis of alcohol use disorder (AUD). Approximately 6.8 percent (16.3 million adults) in the United States over the age of 18 had an AUD in 2014. This includes 10.6 million men and 5.7 million women. In addition, in 2014, an estimated 679,000 adolescents between the ages 12-17 (2.7% of this age group) had an AUD. In 2012, 3.3 million deaths or 5.9 percent of all global deaths (7.6% for men and 4.0% for women) were attributable to alcohol consumption (WHO Global Status Report on Alcohol and Health, 2014).

To be diagnosed with an AUD in the United States, individuals must meet certain criteria outlined in the Diagnostic and Statistical Manual of Mental Disorders (DSM). For example, under the fifth edition of the DSM, any individual meeting two of the eleven criteria during the same 12-month period receives a diagnosis of AUD. The severity of an AUD—mild, moderate, or severe—is based on the number of criteria met. In Europe, individuals are screened using the Alcohol Use Disorders Identification Test (AUDIT). People with AUD drink to excess and, consequently, can endanger both themselves and others.

Alcohol abuse is a drinking pattern that results in significant and recurrent adverse consequences. Alcohol abusers may fail to fulfill major school, work, or family obligations. People with alcoholism (also known as alcohol dependence) have lost reliable control of their alcohol use and are often unable to stop drinking once they start. Alcohol dependence is characterized by tolerance (the need to drink more to achieve the same “high”) and withdrawal symptoms if drinking is suddenly stopped. Withdrawal symptoms may include nausea, sweating, restlessness, irritability, tremors, hallucinations and convulsions.

Problem drinking has multiple causes, with genetic, physiological, psychological, and social factors all playing a role. Not every individual is equally affected by each cause. For some with AUD, psychological traits such as impulsiveness, low self-esteem and a need for approval prompt inappropriate drinking. Genetic factors make some people especially vulnerable to alcohol dependence. AUD can cause physiological changes that make more drinking the only way to avoid discomfort and individuals with AUD may drink partly to reduce or avoid withdrawal symptoms.

People with AUD can seek counseling and psychological therapy from health professionals including physicians, nutritionists, psychiatrists, psychologists, clinical social workers or by attending 12-step Alcoholic Anonymous meetings. However, for a variety of reasons, access to, acceptance of, and success of such resources can be limited.

Considerable resistance to the use of medications for the treatment of AUD persists and current evidence shows that medications are underused in the treatment of AUD. In Europe, oral nalmefene has been approved, and can be taken by a patient while drinking. However, as of January 2015, disulfiram, acamprosate, and oral or extended release injectable naltrexone are the only drugs approved by the Food and Drug Administration in the United States specifically for the treatment of AUD. However, all of these medications must be taken in patients who can abstain from alcohol before the initiation of treatment or have completed alcohol withdrawal. Accordingly, there is still a need for medications which treat subjects with AUD who are still drinking alcohol.

Naltrexone was initially developed to treat opioid dependence due to its effect of blocking the euphoric effects of opioids. Naltrexone tablet formulations for oral administration have been used for treating opioid addiction since 1984. Long-acting depot forms of naltrexone to be administered once monthly or longer were developed in order to improve compliance. Data from clinical trials demonstrated that the depot formulations were effective in reducing relapse to opioid use. Currently, there is one intramuscular, extended-release formulation, and one oral forumulation, of naltrexone (Vivitrol®) for monthly administration approved by the FDA. An intranasal (IN) formulation of naltrexone has the potential to be used for treating AUD without the use of needles or an extended-release formulation. While studies have shown that some opioid antagonists, such as naltrexone, administered in oral or injectable forms, can decrease alcohol drinking and operant responding for it, there remains a substantial need for a simple, fast and compliant means of treating AUD.

### DETAILED DESCRIPTION

Disclosed herein are methods of treatment of alcohol use disorder (AUD) in a subject comprising administering to the subject an IN formulation comprising a therapeutically effective amount of naltrexone and pharmaceutically acceptable salts thereof.

In certain embodiments, the IN formulation is administered prior to ingestion of alcohol. In certain embodiments, the IN formulation is administered about 1-2 hours prior to ingestion of alcohol. In certain embodiments, the IN formulation is administered on a daily basis. In certain embodiments, the IN formulation is administered twice daily. In certain embodiments, the IN formulation is administered three times daily. In certain embodiments, the IN formulation is administered four times daily. In certain embodiments, the IN formulation is administered as needed by the subject throughout the day. In certain embodiments, the IN formulation is administered once daily, followed by additional, subsequent administrations as needed by the subject throughout the day. In certain embodiments, the IN formulation is administered contemporaneously with the ingestion of alcohol. In certain embodiments, the IN formulation is administered following ingestion of alcohol.

In certain embodiments, the IN formulation comprises an aqueous solution. In certain embodiments, the IN formulation comprises about 4 mg naltrexone or a salt thereof. In certain embodiments, about 0.1 mL of said formulation is delivered to the subject. In certain embodiments, the formulation comprising 40 mg/mL naltrexone or a salt thereof.

In certain embodiments, the IN formulation is administered as a single administration to one nostril. In certain embodiments, the IN formulation is administered as two administrations, one to each nostril. In certain embodiments, the IN formulation is administered as four administrations, two to each nostril.

In certain embodiments, the IN formulation comprising a therapeutically effective amount of naltrexone is administered in conjunction with naloxone.

In certain embodiments, the IN formulation additionally comprises an absorption enhancer. In certain embodiments, the absorption enhancer is selected from the group consisting of benzalkonium chloride, chitosan, cyclodextrins, deoxycholic acid, dodecyl maltoside, glycocholic acid, laureth-9, taurocholic acid, and taurodihydrofusidic acid. In certain embodiments, the absorption enhancer is Intravail® alkyl saccharide.

In certain embodiments, the IN formulation additionally comprises one or more excipients selected from sodium chloride, benzalkonium chloride, edetate disodium, and an acid. In certain embodiments, the acid is sufficient to achieve a pH of 3.5-5.5.

In certain embodiments, the therapeutically effective amount comprises about 4 to about 16 mg of naltrexone. In certain embodiments, the therapeutically effective amount comprises about 4, about 5, about 6, about 7, about 8, about 9, about 10, about 11, about 12, about 13, about 14, about 15, or about 16 mg of naltrexone per day.

In certain embodiments, the therapeutically effective amount of naltrexone is administered in 4 mg doses throughout the day as needed by the subject.

In certain embodiments, the therapeutically effective amount of naltrexone is administered as a first 4 mg dose in the morning, and subsequent 4 mg doses as needed prior to consumption of alcohol. In certain embodiments, the therapeutically effective amount of naltrexone is administered as a first 4 mg dose in the morning, and subsequent 4 mg doses as needed contemporaneously with consumption of alcohol. In certain embodiments, the therapeutically effective amount of naltrexone is administered as a first 4 mg dose in the morning, and subsequent 4 mg doses as needed after consumption of alcohol.

Also disclosed herein is a device adapted for nasal delivery of a pharmaceutical composition to a subject suffering AUD, comprising a therapeutically effective amount of naltrexone and pharmaceutically acceptable salts thereof. In certain embodiments, the device is pre-primed. In certain embodiments, the device can be primed before use. In certain embodiments, the device is a single-dose device. In certain embodiments, the device is a multi-dose device.

As use herein, the following terms have the meanings indicated.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “absorption enhancer,” as used herein, refers to a functional excipient included in formulations to improve the absorption of a pharmacologically active drug. This term usually refers to an agent whose function is to increase absorption by enhancing membrane permeation, rather than increasing solubility. As such, such agents are sometimes called permeation enhancers. Examples of absorption enhancers include aprotinin, benzalkonium chloride, benzyl alcohol, capric acid, ceramides, cetylpyridinium chloride, chitosan, cyclodextrins, deoxycholic acid, decanoyl carnitine, dodecyl maltoside, EDTA, glycocholic acid, glycodeoxycholic acid, glycofurol, glycosylated sphingosines, glycyrrhetinic acids, 2-hydroxypropyl-β-cyclodextrin, laureth-9, lauric acid, lauroyl carnitine, sodium lauryl sulfate, lysophosphatidylcholine, menthol, poloxamer 407 or F68, poly-L-arginine, polyoxyethylene-9-lauryl ether, polysorbate 80, propylene glycol, quillaja saponin, salicylic acid, sodium salt, β-sitosterol-β-D-glucoside, sucrose cocoate, taurocholic acid, taurodeoxycholic acid, taurodihydrofusidic acid, and tetradecyl maltoside. Alkylsaccharides (e.g., nonionic alkylsaccharide surfactants such as alkylglycosides and sucrose esters of fatty acids that consist of an aliphatic hydrocarbon chain coupled to a sugar moiety by a glycosidic or ester bond, respectively), cyclodextrins (cyclic oligosaccharides composed of six or more monosaccharide units with a central cavity, which form inclusion complexes with hydrophobic molecules and they have primarily been used to increase drug solubility and dissolution and to enhance low molecular weight drug absorption), chitosans (linear cationic polysaccharides produced from the deacetylation of chitin), and bile salts and their derivatives (such as sodium glycocholate, sodium taurocholate, and sodium taurodihydrofusidate) tend to be amongst the best-tolerated absorption enhancers. See, e.g., Aungst, AAPS Journal 14 (1): 10-8, 2011; Maggio, J. Excipients and Food Chem. 5 (2): 100-12, 2014.

The term “agonist,” as used herein, refers to a moiety that interacts with and activates a receptor, and thereby initiates a physiological or pharmacological response characteristic of that receptor. The term “antagonist,” as used herein, refers to a moiety that competitively binds to a receptor at the same site as an agonist (for example, the endogenous ligand), but which does not activate the intracellular response initiated by the active form of the receptor and can thereby inhibit the intracellular responses by an agonist or partial agonist. An antagonist does not diminish the baseline intracellular response in the absence of an agonist or partial agonist. The term “inverse agonist” refers to a moiety that binds to the endogenous form of the receptor or to the constitutively activated form of the receptor and which inhibits the baseline intracellular response initiated by the active form of the receptor below the normal base level of activity which is observed in the absence of an agonist or partial agonist.

The term “alcohol use disorder” is defined by criteria set forth the Diagnostic and Statistical Manual of Mental Disorders (DSM, most recent revision, presently DSM-V) in the US, or by similar criteria set forth in corresponding well-accepted standards such as the World Health Organization's ICD (International Statistical Classification of Diseases and Related Health Problems, most recent revision, presently the ICD-10). Related terms and disorders include “alcohol abuse” and “alcohol dependence” (used in DSM-IV), “alcohol harmful use” and “alcohol dependence syndrome” (used in the ICD-10), and alcoholism.

The term “antimicrobial preservative,” as used herein, refers to a pharmaceutically acceptable excipient with antimicrobial properties which is added to a pharmaceutical composition to maintain microbiological stability. Compounds act both as preservatives and stabilizers.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “pharmaceutical composition” is used herein interchangeably with the term “Pharmaceutical formulation,” or just “formulation,” and denotes an active pharmaceutical ingredient (i.e., a drug substance) in combination with at least one pharmaceutically acceptable excipient or carrier.

The term “equivalent,” as used herein refers to a weight of the opioid antagonist naltrexone and pharmaceutically acceptable salts thereof that is equimolar to a specified weight of naltrexone hydrochloride.

The term “excipient,” as used herein refers to a natural or synthetic substance formulated alongside the active ingredient of a medication, included for the purpose of long-term stabilization, bulking up solid formulations, or to confer a therapeutic enhancement on the active ingredient in the final dosage form, such as facilitating drug absorption, reducing viscosity, or enhancing solubility.

The term “therapeutically effective dose,” as used herein refers to a dose that is effective to treat a disease, to decrease one or more observable symptoms of a disease, or to delay onset or progression of or mitigate the symptoms of a more serious condition that often follows after the condition that a patient is currently experiencing. A therapeutically effective dose may, but need not necessarily, completely eliminate all symptoms of the disease.

The term “in need of treatment” and the term “in need thereof” when referring to treatment are used interchangeably and refer to a judgment made by a caregiver (e.g. physician, nurse, nurse practitioner, that a subject will benefit from treatment.

As used herein, two embodiments are “mutually exclusive” when one is defined to be something which is different than the other. For example, an embodiment wherein the amount of naltrexone hydrochloride is specified to be 4 mg is mutually exclusive with an embodiment wherein the amount of naltrexone hydrochloride is specified to be 2 mg. However, an embodiment wherein the amount of naltrexone hydrochloride is specified to be 4 mg is not mutually exclusive with an embodiment in which less than about 10% of said pharmaceutical composition leaves the nasal cavity via drainage into the nasopharynx or externally.

The term “naloxone,” as used herein, refers to a compound of the following structure:

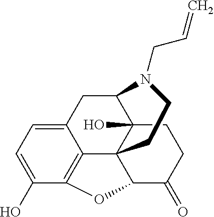

or a pharmaceutically acceptable salt, hydrate, or solvate thereof. The CAS registry number for naloxone is 465-65-6. Other names for naloxone include: 17-allyl-4,5a-epoxy-3,14-dihydroxymorphinan-6-one; (−)-17-allyl-4,5a-epoxy-3,14-dihydroxymorphinan-6-one; 4,5a-epoxy-3, 14-dihydroxy-17-(2-propenyl) morphinan-6-one; and (−)-12-allyl-7,7a,8,9-tetrahydro-3, 7a-dihydroxy-4aH-8,9c-iminoethanophenanthro [4,5-bcd] furan-5 (6H)-one. Naloxone hydrochloride may be anhydrous (CAS Reg. No. 357-08-4) and also forms a dihydrate (CAS No. 51481-60-8). It has been sold under various brand names including Narcan®, Nalone®, Nalossone®, Naloxona®, Naloxonum®, Narcanti®, and Narcon®.

The term “naltrexone,” as used herein, refers to a compound of the following structure:

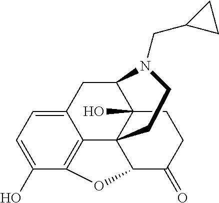

or a pharmaceutically acceptable salt, hydrate, or solvate thereof. The CAS registry number for naltrexone is 16590-41-3. Other names for naltrexone include: 17-(cyclopropylmethyl)-4,5α-epoxy-3, 14-dihydroxymorphinan-6-one; (5a)-17-(cyclopropylmethyl)-3,14-dihydroxy-4,5-epoxymorphinan-6-one; and (1S,5R,13R,17S)-4-(cyclopropylmethyl)-10,17-dihydroxy-12-oxa-4-azapentacyclo [9.6.1.01, 13.05, 17.07, 18] octadeca-7 (18), 8, 10-trien-14-one. Naltrexone hydrochloride (CAS Reg. No. 16676-29-2) has been marketed under the trade names Antaxone®, Depade®, Nalorex®, Revia®, Trexan®, Vivitrex®, and Vivitrol®.

The term “methylnaltrexone,” as used herein, refers to a pharmaceutically acceptable salt comprising the cation (5a)-17-(cyclopropylmethyl)-3,14-dihydroxy-17-methyl-4,5-epoxymorphinanium-17-ium-6-one a compound of the following structure:

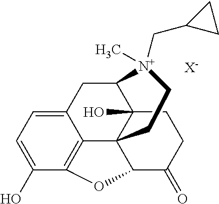

wherein X⁻ is a pharmaceutically acceptable anion. Methylnaltrexone bromide (CAS Reg. No. 75232-52-7) has been marketed under the trade name Relistor®.

The term “nalmefene,” as used herein, refers to 17-cyclopropylmethyl-4,5α-epoxy-6-methylenemorphinan-3,14-diol, a compound of the following structure:

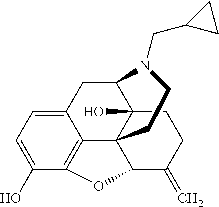

Nalmefene hydrochloride (CAS Reg. No. 58895-64-0) has been marketed under the trade names Nalmetrene®, Cervene®, Revex®, Arthrene®, and Incystene®.

The term “nostril,” as used herein, is synonymous with “naris.”

The term “opioid antagonist” includes, in addition to naltrexone and pharmaceutically acceptable salts thereof: naloxone, methylnaltrexone, and nalmefene, and pharmaceutically acceptable salts thereof. In certain embodiments, the opioid antagonist is naltrexone hydrochloride. In certain embodiments, the opioid antagonist is naltrexone hydrochloride dihydrate. In certain embodiments, the opioid antagonist is naltrexone hydrochloride. In certain embodiments, the opioid antagonist is naloxone. In certain embodiments, the opioid antagonist is methylnaltrexone bromide. In certain embodiments, the nasally administering is accomplished using a device described herein.

The term “pharmaceutical composition,” as used herein, refers to a composition comprising at least one active ingredient; including but not limited to, salts, solvates and hydrates of the opioid antagonist naltrexone, whereby the composition is amenable to use for a specified, efficacious outcome in a mammal (for example, without limitation, a human).

The term “subject,” as used herein, is intended to be synonymous with “patient,” and refers to any mammal (preferably human) afflicted with a condition likely to benefit from a treatment with a therapeutically effective amount of the opioid antagonist naltrexone.

The term “tonicity agent,” as used herein, refers to a compound which modifies the osmolality of a formulation, for example, to render it isotonic. Tonicity agents include, dextrose, lactose, sodium chloride, calcium chloride, magnesium chloride, sorbitol, sucrose, mannitol, trehalose, raffinose, polyethylene glycol, hydroxyethyl starch, glycine and the like.

As used herein, the term “AUC” refers to the area under the drug plasma concentration-time curve. As used herein, the term “AUC₀₋ₜ” refers to the area under the drug plasma concentration-time curve from t=0 to the last measurable concentration. As used herein, the term “AUC_{0-∞}” refers to the area under the drug plasma concentration-time curve extrapolated to ∞. As used herein, the term “AUC_{0-t/D}” refers to the AUC₀₋ₜ normalized to 0.4 mg IM naltrexone. As used herein, the term “AUC_{0-t/D}” refers to the AUC_{0-∞} normalized to 0.4 mg IM naltrexone

As used herein, the term “bioavailability (F)” refers to the fraction of a dose of drug that is absorbed from its site of administration and reaches, in an unchanged form, the systemic circulation. As used herein, the term “absolute bioavailability” is used when the fraction of absorbed drug is related to its IV bioavailability. It may be calculated using the following formula:

$$F = \frac{{AUC}_{extravascular}}{{AUC}_{intravenous}} \times \frac{{Dose}_{intravenous}}{{Dose}_{extravascular}}$$

The term relative bioavailability (Fᵣₑₗ) is used to compare two different extravascular routes of drug administration and it may be calculated using the following formula:

$${F}_{rel} = \frac{{AUC}_{extravascular 1}}{{AUC}_{extravascular 2}} \times \frac{{Dose}_{extravascular 2}}{{Dose}_{extravascular 1}}$$

As used herein, the term “clearance (CL)” refers to the rate at which a drug is eliminated divided by its plasma concentration, giving a volume of plasma from which drug is completely removed per unit of time. CL is equal to the elimination rate constant (λ) multiplied by the volume of distribution (V_{d}), wherein “V_{d}” is the fluid volume that would be required to contain the amount of drug present in the body at the same concentration as in the plasma. As used herein, the term “apparent clearance (CL/F)” refers to clearance that does not take into account the bioavailability of the drug. It is the ratio of the dose over the AUC.

As used herein, the term “Cₘₐₓ” refers to the maximum observed plasma concentration. As used herein, the term “Cₘₐₓ D” refers to Cₘₐₓ normalized to 0.4 mg IM naltrexone.

As used herein, the term “t_{1/2}” or “half-life” refers to the amount of time required for half of a drug to be eliminated from the body or the time required for a drug concentration to decline by half.

As used herein, the term “coefficient of variation (CV)” refers to the ratio of the sample standard deviation to the sample mean. It is often expressed as a percentage.

As used herein, the term “confidence interval” refers to a range of values which will include the true average value of a parameter a specified percentage of the time.

As used herein, the term “elimination rate constant (λ)” refers to the fractional rate of drug removal from the body. This rate is constant in first-order kinetics and is independent of drug concentration in the body. A is the slope of the plasma concentration-time line (on a logarithmic y scale). The term “2z,” as used herein, refers to the terminal phase elimination rate constant, wherein the “terminal phase” of the drug plasma concentration-time curve is a straight line when plotted on a semilogarithmic graph. The terminal phase is often called the “elimination phase” because the primary mechanism for decreasing drug concentration during the terminal phase is drug elimination from the body. The distinguishing characteristic of the terminal elimination phase is that the relative proportion of drug in the plasma and peripheral volumes of distribution remains constant. During this “terminal phase” drug returns from the rapid and slow distribution volumes to the plasma, and is permanently removed from the plasma by metabolism or renal excretion.

### Opioid Antagonists

Opioid receptor antagonists are a well-recognized class of chemical agents. They have been described in detail in the scientific and patent literature. Naltrexone and its active metabolite 6β-naltrexol are opioid antagonists, with no agonist properties, at the μ-opioid receptor (MOR), the κ-opioid receptor (KOR), and the δ-opioid receptor (DOR). Naltrexone operates by reversibly blocking the opioid receptors thereby attenuating the effects of opioids. Its mechanism of action in alcohol dependence is not fully understood but, without being limited by theory, naltrexone likely modulates the dopaminergic mesolimbic pathway (one of the primary centers for risk-reward analysis in the brain, and a tertiary pleasure center) which is believed to be a major center of the reward associated with addiction that all major drugs of abuse are believed to activate. The mechanism of action may be antagonism to endogenous opiates such as tetrahydropapaveroline, whose production is augmented in the presence of alcohol.

Naltrexone is commercially available as a hydrochloride salt. Naltrexone hydrochloride (17-(cyclopropylmethyl)-4,5α-epoxy-3,14-dihydroxymorphinan-6-one) is used to prevent euphorigenic effects in the treatment of patients addicted to opioids. It markedly blocks the physical dependence to intravenously administered opioids and motivates withdrawal from opioid dependency, but the patient does not develop tolerance or dependence to naltrexone. Naltrexone is also effective in reducing the craving for alcohol in the treatment of alcoholism, especially when combined with psychosocial therapy.

When naltrexone hydrochloride is administered intranasally, rather than orally, the bioavailability is significantly higher. When administered orally, despite being nearly completely absorbed from the gastrointestinal tract, naltrexone undergoes rapid and extensive first-pass metabolism to 6-B-naltrexol. As a result, the amount of naltrexone reaching systemic circulation is limited. The oral bioavailability of naltrexone has been reported to be as low as 5%. Gonzalez and Brogden, Drugs 35:192-213, 1988. Studies presented herein found the oral bioavailability of naltrexone to be similarly low, about 9%.

Provided herein are methods of treatment employing nasal delivery of a pharmaceutical composition to a patient, comprising a therapeutically effective amount of the opioid antagonist naltrexone. In certain embodiments, the therapeutically effective amount is equivalent to about 1 to about 16 mg of naltrexone. In certain embodiments, the therapeutically effective amount is equivalent to about 1, about 2, about 3, about 4, about 5, about 6, about 7, about 8, about 9, about 10, about 11, about 12, about 13, about 14, about 15, or about 16 mg of naltrexone. In certain embodiments, the therapeutically effective amount is equivalent to about 4 mg of naltrexone hydrochloride. In certain embodiments, the opioid antagonist is naltrexone hydrochloride. In certain embodiments, the opioid antagonist is anhydrous naltrexone hydrochloride. In certain embodiments, the opioid antagonist is naltrexone hydrochloride dihydrate.

While many of the embodiments of the pharmaceutical compositions described herein will be described and exemplified with naltrexone, other opioid antagonists can be adapted for nasal delivery based on the teachings of the specification. In fact, it should be readily apparent to one of ordinary skill in the art from the teachings herein that the devices and pharmaceutical compositions described herein may be suitable for other opioid antagonists. The opioid receptor antagonists described herein include μ-opioid, k-opioid, and 8-opioid receptor antagonists. Examples of useful opioid receptor antagonists include naltrexone, naloxone, methylnaltrexone, and nalmefene. Other useful opioid receptor antagonists are known in the art (e.g., U.S. Pat. No. 4,987,136).

### Pharmaceutical Formulations

Also provided are pharmaceutical compositions comprising the opioid antagonist naltrexone. In certain embodiments the pharmaceutical compositions comprise the opioid antagonist naltrexone and a pharmaceutically acceptable carrier. The carrier(s) must be “acceptable” in the sense of being compatible with the other ingredients of the formulation and not overly deleterious to the recipient thereof. Some embodiments of the present invention include a method of producing a pharmaceutical composition comprising admixing the opioid antagonist naltrexone and a pharmaceutically acceptable carrier. Pharmaceutical compositions are applied directly to the nasal cavity using the devices described herein. In the case of a spray, this may be achieved for example by means of a metering atomizing spray pump.

Liquid preparations include solutions, suspensions and emulsions, for example, water or water-propylene glycol solutions. Additional ingredients in liquid preparations may include: antimicrobial preservatives, such as benzalkonium chloride, methylparaben, sodium benzoate, benzoic acid, phenyl ethyl alcohol, and the like, and mixtures thereof; surfactants such as Polysorbate 80 NF, polyoxyethylene 20 sorbitan monolaurate, polyoxyethylene (4) sorbitan monolaurate, polyoxyethylene 20 sorbitan monopalmitate, polyoxyethylene 20 sorbitan monostearate, polyoxyethylene (4) sorbitan monostearate, polyoxyethylene 20 sorbitan tristearate, polyoxyethylene (5) sorbitan monooleate, polyoxyethylene 20 sorbitan trioleate, polyoxyethylene 20 sorbitan monoisostearate, sorbitan monooleate, sorbitan monolaurate, sorbitan monopalmitate, sorbitan monostearate, sorbitan trilaurate, sorbitan trioleate, sorbitan tristearate, and the like, and mixtures thereof; a tonicity agent such as: dextrose, lactose, sodium chloride, calcium chloride, magnesium chloride, sorbitol, sucrose, mannitol, trehalose, raffinose, polyethylene glycol, hydroxyethyl starch, glycine, and the like, and mixtures thereof; and a suspending agent such as microcrystalline cellulose, carboxymethylcellulose sodium NF, polyacrylic acid, magnesium aluminum silicate, xanthan gum, and the like, and mixtures thereof.

Ideally, when an opioid antagonist is administered intranasally prior to ingestion of alcohol to treat AUD, the opioid antagonist is absorbed quickly, i.e., within about fifteen to about thirty minutes and/or yielding a time to the maximum plasma concentration (Tₘₐₓ) of about 25 to about 40 minutes. For example, in certain embodiments, the opioid antagonist is absorbed within the first 15 min after administration and the time to the maximum plasma concentration (Tₘₐₓ) is 25 min or less. Alternatively, the opioid antagonist is absorbed within the first 30 min after administration and the Tₘₐₓ is 40 min or less.

The use of absorption enhancers, such as alkylsaccharides, cyclodextrins, and chitosans may increase the rate at which naltrexone is absorbed and decrease the Tₘₐₓ. Such absorption enhancers typically operate by affecting two primary mechanisms for nasal absorption: paracellular transport via opening of tight junctions between cells, and transcellular transport or transcytosis through cells via vesicle carriers.

For example, Intravail® is the alkyl saccharide 1-O-n-dodecyl-β-D-maltopyranoside (alternately referred to as lauryl-β-D-maltopyranoside, dodecyl maltopyranoside, dodecyl maltoside, and DDM; C₂₄H₄₆Q₁₁). Alkylsaccharides are used in commercial food and personal care products and have been designated Generally Recognized as Safe (GRAS) substances for food applications. They are non-irritating enhancers of transmucosal absorption that are odorless, tasteless, non-toxic, non-mutagenic, and non-sensitizing in the Draize test up to a 25% concentration. Alkylsaccharides increase absorption by increasing paracellular permeability, as indicated by a decrease in transepithelial electrical resistance; they may also increase transcytosis. The effect is short-lived. Other alkylsaccharides include tetradecyl maltoside (TDM) and sucrose dodecanoate.

In certain embodiments, an intranasal formulation comprises about 0.05% to about 2.5% Intravail. In certain embodiments, an intranasal formulation comprises about 0.1% to about 0.5% Intravail®. In certain embodiments, an intranasal formulation comprises about 0.15% to about 0.35% Intravail®. In certain embodiments, an intranasal formulation comprises about 0.15% to about 0.2% Intravail®. In certain embodiments, an intranasal formulation comprises about 0.18% Intravail®. In certain embodiments, an intranasal formulation comprises about 0.2% to about 0.3% Intravail®. In certain embodiments, an intranasal formulation comprises about 0.25% Intravail®.

When 0.18% Intravail® was added to an intranasal formulation of sumatriptan, the maximum plasma concentration increased almost four-fold in comparison to Imitrex nasal spray and Tₘₐₓ was reduced from 1-2 hours to 8-10 minutes. Total exposure, as measured by the area under the concentration-time curve (AUC), increased 32%. An intranasal formulation of naltrexone has the potential to be used for treating AUD without the use of needles or an extended-release formulation. Inclusion of Intravail® may improve pharmacokinetic parameters in some applications.

Some absorption enhancing excipients can alter the paracellular and/or transcellular pathways, others can extend residence time in the nasal cavity or prevent metabolic changes. Without an absorption enhancer, the molecular-weight limit for nasal absorption is about 1 kDa, while administration of drugs in conjunction with absorption enhancers can enable the absorption of molecules from 1-30 kDa. Intranasal administration of most absorption enhancers, however, can cause nasal mucosa damage. Maggio, J. Excipients and Food Chem. 5 (2): 100-12, 2014.

Examples of absorption enhancers include aprotinin, benzalkonium chloride, benzyl alcohol, capric acid, ceramides, cetylpyridinium chloride, chitosan, cyclodextrins, deoxycholic acid, decanoyl carnitine, dodecyl maltoside, EDTA, glycocholic acid, glycodeoxycholic acid, glycofurol, glycosylated sphingosines, glycyrrhetinic acids, 2-hydroxypropyl-β-cyclodextrin, laureth-9, lauric acid, lauroyl carnitine, lauryl sulfate, lysophosphatidylcholine, menthol, poloxamer 407, poloxamer F68, poly-L-arginine, polyoxyethylene-9-lauryl ether, polysorbate 80, propylene glycol, quillaja saponin, salicylic acid, β-Sitosterol-β-D-glucoside, sucrose cocoate, taurocholic acid, taurodeoxycholic acid, taurodihydrofusidic acid, and tetradecyl maltoside.

The opioid antagonist naltrexone described herein can be formulated into pharmaceutical compositions using techniques well known to those in the art. Suitable pharmaceutically acceptable carriers, outside those mentioned herein, are known in the art.

The opioid antagonist naltrexone described herein may optionally exist as pharmaceutically acceptable salts including pharmaceutically acceptable acid addition salts prepared from pharmaceutically acceptable non-toxic acids including inorganic and organic acids. Representative acids include, but are not limited to, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic and the like, such as those pharmaceutically acceptable salts listed by Berge et al., Journal of Pharmaceutical Sciences, 66:1-19 (1977). The acid addition salts may be obtained as the direct products of compound synthesis. In the alternative, the free base may be dissolved in a suitable solvent containing the appropriate acid and the salt isolated by evaporating the solvent or otherwise separating the salt and solvent. The opioid antagonist naltrexone described herein may form solvates with standard low molecular weight solvents using methods known to the skilled artisan.

Accordingly, provided herein are pharmaceutical formulations for intranasal administration comprising naltrexone. In certain embodiments, the formulation is an aqueous solution. In certain embodiments, the formulation comprises, per dose, between about 25 and about 200 μL of the aqueous solution. In certain embodiments, the formulation comprises, per dose, between about 50 and about 200 μL of the aqueous solution. In certain embodiments, the formulation comprises, per dose, not more than about 140 μL. In certain embodiments, the formulation comprises, per dose, not more than about 100 μL.

In certain embodiments, the formulation comprises between about 1% (w/v) and about 16% (w/v) of the opioid antagonist naltrexone. In certain embodiments, the formulation comprises between about 2% (w/v) and about 12% (w/v) of naltrexone. In certain embodiments, the formulation comprises between about 2% (w/v) and about 10% (w/v) of naltrexone. In certain embodiments, the formulation comprises between about 2% (w/v) and about 8% (w/v) of naltrexone. In certain embodiments, the formulation comprises between about 2% (w/v) and about 4% (w/v) of naltrexone. In certain embodiments, the formulation comprises about 1% (w/v), about 2% (w/v), about 3% (w/v), about 4% (w/v), about 5% (w/v), about 6% (w/v), about 7% (w/v), or about 8% (w/v) of naltrexone. In certain embodiments, the formulation comprises about 1% (w/v) of naltrexone. In certain embodiments, the formulation comprises about 2% (w/v) of naltrexone. In certain embodiments, the formulation comprises about 4% (w/v) of naltrexone.

In certain embodiments, the formulation comprises between about 1 mg and about 16 mg of the opioid antagonist naltrexone. In certain embodiments, the formulation comprises between about 2 mg and about 12 mg of naltrexone. In certain embodiments, the formulation comprises between about 2 mg and about 10 mg of naltrexone. In certain embodiments, the formulation comprises between about 2 mg and about 8 mg of naltrexone. In certain embodiments, the formulation comprises between about 2 mg and about 4 mg of naltrexone. In certain embodiments, the formulation comprises about 1 mg, about 2 mg, about 3 mg, about 4 mg, about 5 mg, about 6 mg, about 7 mg, or about 8 mg of naltrexone. In certain embodiments, the formulation comprises about 1 mg of naltrexone. In certain embodiments, the formulation comprises about 2 mg of naltrexone. In certain embodiments, the formulation comprises about 4 mg of naltrexone.

In certain embodiments, provided herein are pharmaceutical formulations for intranasal administration comprising, in an aqueous solution of not more than about 140 μL:
between about 2 mg and about 16 mg of naltrexone; and
between about 0.2 mg and about 1.2 mg of an isotonicity agent.

In certain embodiments, provided herein are pharmaceutical formulations for intranasal administration comprising, in an aqueous solution of not more than about 140 μL: between about 2% (w/v) and about 16% (w/v) of naltrexone; and
between about 0.2% (w/v) and about 1.2% (w/v) of an isotonicity agent.

In certain embodiments, the pharmaceutical formulation comprises:
about 2 mg or about 4 mg naltrexone hydrochloride or a hydrate thereof; and between about 0.2 mg and about 1.2 mg of an isotonicity agent.

In certain embodiments, the pharmaceutical formulation comprises:
about 2% (w/v) or about 4% (w/v) naltrexone hydrochloride or a hydrate thereof; and
between about 0.2% (w/v) and about 1.2% (w/v) of an isotonicity agent.

In certain embodiments, the isotonicity agent is sodium chloride.

In certain embodiments, the pharmaceutical formulation comprises:
about 2 mg or about 4 mg naltrexone hydrochloride; and
about 0.74 mg sodium chloride.

In certain embodiments, the pharmaceutical formulation comprises:
about 4 mg naltrexone hydrochloride; and
about 0.74 mg sodium chloride.

In certain embodiments, provided herein are pharmaceutical formulations above comprise an aqueous solution of not more than about 100 μL

In certain embodiments, the pharmaceutical formulation comprises about 4 mg or about 4% (w/v) naltrexone hydrochloride or a hydrate thereof. In certain embodiments, the pharmaceutical formulation comprises about 2 mg or about 2% (w/v) naltrexone hydrochloride or a hydrate thereof. In certain embodiments, the naltrexone hydrochloride is provided as naltrexone hydrochloride dihydrate.

In certain embodiments, the pharmaceutical formulation additionally comprises an absorption enhancer. In certain embodiments, the pharmaceutical formulation comprises between about 0.005% to about 2.5% of the absorption enhancer. In certain embodiments, the pharmaceutical formulation comprises between about 0.05% to about 2.5% of the absorption enhancer. In certain embodiments, the pharmaceutical formulation comprises between about 0.1% to about 0.5% of the absorption enhancer. In certain embodiments, the pharmaceutical formulation comprises about 0.25% of the absorption enhancer. In certain embodiments, the pharmaceutical formulation comprises about 0.18% of the absorption enhancer. In certain embodiments, the absorption enhancer is an alkylsaccharide. In certain embodiments, the alkylsaccharide is chosen from dodecyl maltoside, tetradecyl maltoside (TDM) and sucrose dodecanoate. In certain embodiments, the alkylsaccharide is Intravail (dodecyl maltoside). In certain embodiments, the pharmaceutical formulation comprises between about 0.005% to about 0.05% of the absorption enhancer. In certain embodiments, the pharmaceutical formulation comprises between about 0.005% to about 0.015% of the absorption enhancer. In certain embodiments, the pharmaceutical formulation comprises about 0.01% of the absorption enhancer. In certain embodiments, the absorption enhancer is benzalkonium chloride.

In certain embodiments, the pharmaceutical formulation additionally comprises an isotonicity agent.

In certain embodiments, provided herein are pharmaceutical formulations for intranasal administration comprising, in an aqueous solution of not more than about 140 μL:
between about 2 mg and about 16 mg of naltrexone;
about 0.05 mg to about 2.5 mg of an absorption enhancer; and
between about 0.2 mg and about 1.2 mg of an isotonicity agent.

In certain embodiments, provided herein are pharmaceutical formulations for intranasal administration comprising, in an aqueous solution of not more than about 140 μL:
between about 2% (w/v) and about 16% (w/v) of naltrexone;
about 0.05% (w/v) to about 2.5% (w/v) of an absorption enhancer; and between about 0.2% (w/v) and about 1.2% (w/v) of an isotonicity agent.

In certain embodiments, the pharmaceutical formulation comprises:
about 2 mg or about 4 mg naltrexone hydrochloride or a hydrate thereof;
about 0.05 mg to about 2.5 mg of an absorption enhancer; and between about 0.2 mg and about 1.2 mg of an isotonicity agent.

In certain embodiments, the pharmaceutical formulation comprises:
about 2% (w/v) or about 4% (w/v) naltrexone hydrochloride or a hydrate thereof;
about 0.05% (w/v) to about 2.5% (w/v) of an absorption enhancer; and
between about 0.2% (w/v) and about 1.2% (w/v) of an isotonicity agent.

In certain embodiments, provided herein are pharmaceutical formulations above comprise an aqueous solution of not more than about 100 μL

In certain embodiments, provided herein are pharmaceutical formulations for intranasal administration comprising, in an aqueous solution of not more than about 140 μL:
between about 2 mg and about 16 mg of naltrexone;
about 0.005 mg to about 0.015 mg of an absorption enhancer; and
between about 0.2 mg and about 1.2 mg of an isotonicity agent.

In certain embodiments, provided herein are pharmaceutical formulations for intranasal administration comprising, in an aqueous solution of not more than about 140 μL:
between about 2% (w/v) and about 16% (w/v) of naltrexone;
about 0.005% (w/v) to about 0.015% (w/v) of an absorption enhancer; and
between about 0.2% (w/v) and about 1.2% (w/v) of an isotonicity agent.

In certain embodiments, the pharmaceutical formulation comprises:
about 2 mg or about 4 mg naltrexone hydrochloride or a hydrate thereof;
about 0.005 mg to about 0.015 mg of an absorption enhancer; and
between about 0.2 mg and about 1.2 mg of an isotonicity agent.

In certain embodiments, the pharmaceutical formulation comprises:
about 2% (w/v) or about 4% (w/v) naltrexone hydrochloride or a hydrate thereof;
about 0.005% (w/v) to about 0.015% (w/v) of an absorption enhancer; and
between about 0.2% (w/v) and about 1.2% (w/v) of an isotonicity agent.

In certain embodiments, provided herein are pharmaceutical formulations above comprise an aqueous solution of not more than about 100 μL

In certain embodiments, the isotonicity agent is sodium chloride.

In certain embodiments, the absorption enhancer is Intravail® (dodecyl maltoside).

In certain embodiments, the pharmaceutical formulation comprises:
about 2 mg or about 4 mg naltrexone hydrochloride;
about 0.25 mg Intravail® (dodecyl maltoside); and
about 0.74 mg sodium chloride.

In certain embodiments, the pharmaceutical formulation comprises:
about 4 mg naltrexone hydrochloride;
about 0.25 mg Intravail® (dodecyl maltoside); and
about 0.74 mg sodium chloride.

In certain embodiments, the absorption enhancer is benzalkonium chloride.

In certain embodiments, the pharmaceutical formulation comprises:
about 2 mg or about 4 mg naltrexone hydrochloride;
about 0.01 mg benzalkonium chloride; and
about 0.74 mg sodium chloride.

In certain embodiments, the pharmaceutical formulation comprises:
about 4 mg naltrexone hydrochloride;
about 0.01 mg benzalkonium chloride; and
about 0.74 mg sodium chloride.

In certain embodiments, provided herein are pharmaceutical formulations above comprise an aqueous solution of not more than about 100 μL

In certain embodiments, the pharmaceutical formulation comprises about 4 mg or about 4% (w/v) naltrexone hydrochloride or a hydrate thereof. In certain embodiments, the pharmaceutical formulation comprises about 2 mg or about 2% (w/v) naltrexone hydrochloride or a hydrate thereof. In certain embodiments, the naltrexone hydrochloride is provided as naltrexone hydrochloride dihydrate.

In certain embodiments, the pharmaceutical formulation additionally comprises a compound which is a preservative and/or surfactant.

In certain embodiments, the preservative and/or surfactant is chosen from benzalkonium chloride, methylparaben, sodium benzoate, benzoic acid, phenyl ethyl alcohol, and the like, and mixtures thereof; surfactants such as Polysorbate 80 NF, polyoxyethylene 20 sorbitan monolaurate, polyoxyethylene (4) sorbitan monolaurate, polyoxyethylene 20 sorbitan monopalmitate, polyoxyethylene 20 sorbitan monostearate, polyoxyethylene (4) sorbitan monostearate, polyoxyethylene 20 sorbitan tristearate, polyoxyethylene (5) sorbitan monooleate, polyoxyethylene 20 sorbitan trioleate, polyoxyethylene 20 sorbitan monoisostearate, sorbitan monooleate, sorbitan monolaurate, sorbitan monopalmitate, sorbitan monostearate, sorbitan trilaurate, sorbitan trioleate, sorbitan tristearate, and the like, and mixtures thereof.

In certain embodiments, the pharmaceutical formulation additionally comprises a stabilizing agent.

In certain embodiments, the stabilizing agent is disodium edetate (EDTA).

Also provided herein are pharmaceutical formulations for intranasal administration comprising, in an aqueous solution of not more than about 200 μL:
between about 2 mg and about 16 mg of naltrexone;
between about 0.2 mg and about 1.2 mg of an isotonicity agent;
optionally, between about 0.005 mg and about 0.015 mg of a compound which is a preservative and/or cationic surfactant;
optionally, between about 0.005% to about 2.5% of an absorption enhancer;
optionally, between about 0.1 mg and about 0.5 mg of a stabilizing agent; and
an amount of an acid sufficient to achieve a pH of 3.5-5.5.

In certain embodiments, the pharmaceutical formulation comprises:
between about 2 mg and about 16 mg of naltrexone;
between about 0.2 mg and about 1.2 mg of an isotonicity agent;
between about 0.005 mg and about 0.015 mg of a compound which is a preservative and/or cationic surfactant;
between about 0.005 and about 0.70 mg of a compound which is an absorption enhancer;
between about 0.1 mg and about 0.5 mg of a stabilizing agent; and
an amount of an acid sufficient to achieve a pH of 3.5-5.5.

In certain embodiments, the pharmaceutical formulation comprises:
about 2 mg or about 4 mg naltrexone hydrochloride or a hydrate thereof;
between about 0.2 mg and about 1.2 mg of an isotonicity agent;
between about 0.005 mg and about 0.015 mg of a compound which is a preservative and/or cationic surfactant;
between about 0.005 and about 0.70 mg of a compound which is an absorption enhancer;
between about 0.1 mg and about 0.5 mg of a stabilizing agent;
an amount of hydrochloric acid sufficient to achieve a pH of 3.5-5.5.

In certain embodiments, the isotonicity agent is sodium chloride. In certain embodiments, the preservative and/or cationic surfactant is benzalkonium chloride. In certain embodiments, the absorption enhancer is selected from the group consisting of benzalkonium chloride, chitosan, cyclodextrins, deoxycholic acid, dodecyl maltoside, glycocholic acid, laureth-9, taurocholic acid, and taurodihydrofusidic acid. In certain embodiments, the absorption enhancer is Intravail®. In certain embodiments, the stabilizing agent is edetate disodium. In certain embodiments, the acid is hydrochloric acid.

In certain embodiments, the pharmaceutical formulation comprises:
about 2 mg or about 4 mg naltrexone hydrochloride or a hydrate thereof;
about 0.74 mg sodium chloride;
about 0.01 mg benzalkonium chloride;
about 0.25 mg Intravail® (dodecyl maltoside);
about 0.2 mg edetate disodium; and
an amount of hydrochloric acid sufficient to achieve a pH of 3.5-5.5.

In certain embodiments, the pharmaceutical formulation comprises:
about 2 mg or about 4 mg naltrexone hydrochloride or a hydrate thereof;
about 0.74 mg sodium chloride;
about 0.01 mg benzalkonium chloride;
about 0.2 mg edetate disodium; and
an amount of hydrochloric acid sufficient to achieve a pH of 3.5-5.5.

In certain embodiments, the pharmaceutical formulation comprises:
about 2 mg or about 4 mg naltrexone hydrochloride or a hydrate thereof;
about 0.74 mg sodium chloride;
about 0.25 mg Intravail® (dodecyl maltoside);
about 0.2 mg edetate disodium; and
an amount of hydrochloric acid sufficient to achieve a pH of 3.5-5.5.

In certain embodiments, the pharmaceutical formulation comprises about 4 mg naltrexone hydrochloride or a hydrate thereof. In certain embodiments, the pharmaceutical formulation comprises between about 2.5 mg and about 8 mg naltrexone hydrochloride or a hydrate thereof. In certain embodiments, the pharmaceutical formulation comprises about 2 mg naltrexone hydrochloride or a hydrate thereof. In certain embodiments, the pharmaceutical formulation comprises about 2.5 mg naltrexone hydrochloride or a hydrate thereof. In certain embodiments, the pharmaceutical formulation comprises about 4 mg naltrexone hydrochloride dihydrate.

Also provided herein are pharmaceutical formulations for intranasal administration comprising, in an aqueous solution of about 100 μL:
about 4 mg naltrexone hydrochloride or a hydrate thereof;
between about 0.2 mg and about 1.2 mg of an isotonicity agent;
between about 0.005 mg and about 0.015 mg of a compound which is a preservative and/or cationic surfactant;
between about 0.00 and about 0.50 mg of a compound which is an absorption enhancer;
between about 0.1 mg and about 0.5 mg of a stabilizing agent; and an amount of an acid sufficient to achieve a pH of 3.5-5.5.

Also provided herein are pharmaceutical formulations for intranasal administration comprising, in an aqueous solution of about 100 μL:
about 4 mg naltrexone hydrochloride or a hydrate thereof;
between about 0.2 mg and about 1.2 mg of an isotonicity agent;
optionally, between about 0.005 mg and about 0.015 mg of a compound which is a preservative and/or cationic surfactant;
between about 0.005 and about 0.50 mg of a compound which is an absorption enhancer;
optionally, between about 0.1 mg and about 0.5 mg of a stabilizing agent; and
an amount of an acid sufficient to achieve a pH of 3.5-5.5.

Also provided herein are pharmaceutical formulations for intranasal administration comprising, in an aqueous solution of about 100 μL:
about 4 mg naltrexone hydrochloride or a hydrate thereof;
between about 0.2 mg and about 1.2 mg of an isotonicity agent;
between about 0.005 mg and about 0.015 mg of a compound which is a preservative and/or cationic surfactant;
between about 0.05 and about 0.50 mg of a compound which is an absorption enhancer;
between about 0.1 mg and about 0.5 mg of a stabilizing agent; and
an amount of an acid sufficient to achieve a pH of 3.5-5.5.

In certain embodiments, the pharmaceutical formulation comprises:
about 4 mg naltrexone hydrochloride or a hydrate thereof;
about 0.74 mg sodium chloride;
about 0.01 mg benzalkonium chloride;
about 0.18 mg Intravail® (dodecyl maltoside);
about 0.2 mg edetate disodium; and
an amount of hydrochloric acid sufficient to achieve a pH of 3.5-5.5.

Also provided herein are pharmaceutical formulations for intranasal administration comprising, in an aqueous solution of about 100 μL:
about 2 mg naltrexone hydrochloride or a hydrate thereof;
between about 0.2 mg and about 1.2 mg of an isotonicity agent;
between about 0.005 mg and about 0.015 mg of a compound which is a preservative and/or cationic surfactant;
between about 0.00 and about 0.50 mg of a compound which is an absorption enhancer;
between about 0.1 mg and about 0.5 mg of a stabilizing agent; and
an amount of an acid sufficient to achieve a pH of 3.5-5.5.

In certain embodiments, the pharmaceutical formulation comprises:
about 2 mg naltrexone hydrochloride dihydrate;
about 0.74 mg sodium chloride;
about 0.01 mg benzalkonium chloride;
about 0.18 mg Intravail® (dodecyl maltoside);
about 0.2 mg edetate disodium; and
an amount of hydrochloric acid sufficient to achieve a pH of 3.5-5.5.

In certain embodiments, said opioid antagonist is chosen from naltrexone, naloxone, and nalmefene. In certain embodiments, said opioid antagonist is naltrexone hydrochloride, or a hydrate thereof. In certain embodiments, said opioid antagonist is naltrexone hydrochloride dihydrate. In certain embodiments, the opioid antagonist is methylnaltrexone bromide. In certain embodiments, the opioid antagonist is naloxone. In certain embodiments, the opioid antagonist is nalmefene hydrochloride.

In certain embodiments, the therapeutically effective amount comprises about 2 to about 16 mg of naltrexone. In certain embodiments, the pharmaceutical formulation comprises an amount equivalent to about 2, about 3, about 4, about 5, about 6, about 7, about 8, about 9, about 10, about 11, about 12, about 13, about 14, about 15, or about 16 mg of naltrexone hydrochloride. In certain embodiments, the pharmaceutical formulation comprises an amount equivalent to about 4 mg to about 8 mg of naloxone hydrochloride. In certain embodiments, the pharmaceutical formulation comprises an amount equivalent to about 16 mg of naloxone hydrochloride.

In certain embodiments, the pharmaceutical composition is in an aqueous solution of about 100 μL.

In certain embodiments, upon nasal delivery of said pharmaceutical composition to said patient, less than about 10% of said pharmaceutical composition leaves the nasal cavity via drainage into the nasopharynx or externally.

### Nasal Drug Delivery Devices and Kits

Also provided are pharmaceutical compositions in a device adapted for nasal delivery to a subject suffering AUD, comprising a therapeutically effective amount of the opioid antagonist naltrexone and pharmaceutically acceptable salts thereof. In certain embodiments, the device is pre-primed. In certain embodiments, the device can be primed before use. In certain embodiments, the device can be actuated with one hand.

Nasal delivery is considered an attractive route for systemic drug delivery, especially when rapid absorption and effect are desired. In addition, nasal delivery may help address issues related to unpleasant taste, poor bioavailability, slow absorption, drug degradation, adverse events (AEs) in the gastrointestinal tract, and avoids first-pass metabolism and the hepatic toxicity associated with long-term oral naltrexone usage.

Liquid nasal formulations are mainly aqueous solutions, but suspensions and emulsions can also be delivered.

Some emergency medical service (EMS) programs have developed a system using existing technologies of an approved drug and an existing medical device to administer the opioid antagonist naloxone intranasally, albeit in a non-FDA approved manner. This has been accomplished by using the injectable formulation (1 mg/mL) and administering 1 mL per nostril via a marketed nasal atomizer/nebulizer device. The system combines an FDA-approved naloxone injection product (with a Luer fitted tip, no needles) with a marketed, medical device called the Mucosal Atomization Device (MAD™ Nasal, Wolfe Tory Medical, Inc.). This initiative is consistent with the U.S. Needlestick Safety and Prevention Act (Public Law 106-430). The EMS programs recognize limitations of this system, one limitation being that it is not assembled and ready-to-use. Although this administration mode appears to be effective in reversing narcosis, the formulation is not concentrated for retention in the nasal cavity. The 1 mL delivery volume per nostril is larger than that generally utilized for intranasal drug administration. Therefore, there is loss of drug from the nasal cavity, due either to drainage into the nasopharynx or externally from the nasal cavity. The devices described herein are improved ready-to-use products specifically optimized, concentrated, and formulated for nasal delivery.

Metered spray pumps have dominated the nasal drug delivery market since they were introduced. The pumps typically deliver 100 μL (or other volumes in the range of 25-200 μL, and higher) per spray, and they offer high reproducibility of the emitted dose and plume geometry in in vitro tests.

Examples of standard metered spray pumps include those offered by Aptar Pharma, Inc., such as the multi-dose “classic technology platform” nasal spray devices. Such devices comprise a reservoir which holds multiple doses of the nasal spray formulation (e.g., 50, 100, 150, 200, 60, or 120 doses), a closure (e.g., screw, crimp, or snap-on), and an actuator which delivers anywhere from 45 to 1000 μL (e.g. 50, 100, 140, 150, or 200 μL) of fluid per actuation to comprise a single dose. The actuator may be configured to count doses, deliver gel formulations, deliver in an upside-down configuration, etc.

In traditional spray pump systems, antimicrobial preservatives are typically required to maintain microbiological stability in liquid formulations. However, preservative-free systems are also available, e.g. the Advanced Preservative Free (APF) system from Aptar, which is vented, contains a filter membrane for air flow which prevents contamination, has a metal-free fluid path for oxidizing formulations, and can be used in any orientation. Additional nasal spray devices from Aptar and others are optimized with dispenser tips that prevent clogging (useful for high-viscosity and high-volatile formulations), actuators that do not need re-priming after long periods of disuse, etc.

The particle size and plume geometry can vary within certain limits and depend on the properties of the pump, the formulation, the orifice of the actuator, and the force applied. The droplet size distribution of a nasal spray is a critical parameter, since it significantly influences the in vivo deposition of the drug in the nasal cavity. The droplet size is influenced by the actuation parameters of the device and the formulation. The prevalent median droplet size should be between about 30 and about 100 μm. If the droplets are too large (>about 120 μm), deposition takes place mainly in the anterior parts of the nose, and if the droplets are too small (<about 10 μm), they can possibly be inhaled and reach the lungs, which should be avoided because of safety reasons. In its capacity as a surfactant, benzalkonium chloride can affect the surface tension of droplets from a delivered nasal spray plume, producing spherical or substantially spherical particles having a narrow droplet size distribution (DSD), as well as the viscosity of a liquid formulation.

Plume geometry, droplet size and DSD of the delivered plume subsequent to spraying may be measured under specified experimental and instrumental conditions by appropriate and validated and/or calibrated analytical procedures known in the art. These include photography, laser diffraction, and impaction systems (cascade impaction, NGI). Plume geometry, droplet size and DSD can affect pharmacokinetic outcomes such as Cₘₐₓ, Tₘₐₓ, and linear dose proportionality.

Droplet size distribution can be controlled in terms of ranges for the D10, D50, D90, span [(D90-D10)/D50], and percentage of droplets less than 10 mm. In certain embodiments, the formulation will have a narrow DSD. In certain embodiments, the formulation will have a D(v,50) of 30-70 μm and a D(v, 90)<100 μm.

In certain embodiments, the percent of droplets less than 10 μm will be less than 10%. In certain embodiments, the percent of droplets less than 10 μm will be less than 5%. In certain embodiments, the percent of droplets less than 10 μm will be less than 2%. In certain embodiments, the percent of droplets less than 10 μm will be less than 1%.

In certain embodiments, the formulation when dispensed by actuation from the device will produce a uniform circular plume with an ovality ratio close to 1. Ovality ratio is calculated as the quotient of the maximum diameter (Dₘₐₓ) and the minimum diameter (Dₘᵢₙ) of a spray pattern taken orthogonal to the direction of spray flow (e.g., from the “top”). In certain embodiments, the ovality ratio is less than ±2.0. In certain embodiments, the ovality ratio is less than ±1.5. In certain embodiments, the ovality ratio is less than ±1.3. In certain embodiments, the ovality ratio is less than ±1.2. In certain embodiments, the ovality ratio is less than ±1.1. In certain embodiments, the ovality ratio is about ±1.0.

The details and mechanical principles of particle generation for different types of nasal aerosol devices has been described. Reviewed in Vidgren and Kublik, Adv. Drug Deliv. Rev. 29:157-77, 1998. Traditional spray pumps replace the emitted liquid with air, and preservatives are therefore required to prevent contamination. However, driven by the studies suggesting possible negative effects of preservatives (e.g., irritation of nasal mucosa), pump manufacturers have developed different spray systems that avoid the need for preservatives. These systems use a collapsible bag, a movable piston, or a compressed gas to compensate for the emitted liquid volume (www.aptar.com and www.rexam.com). The solutions with a collapsible bag and a movable piston compensating for the emitted liquid volume offer the additional advantage that they can be emitted upside down, without the risk of sucking air into the dip tube and compromising the subsequent spray. This may be useful for some products where the patients are bedridden and where a head-down application is recommended. Another method used for avoiding preservatives is that the air that replaces the emitted liquid is filtered through an aseptic air filter. In addition, some systems have a ball valve at the tip to prevent contamination of the liquid inside the applicator tip (www.aptar.com). More recently, pumps have been designed with side-actuation and introduced for delivery of fluticasone furoate for the indication of seasonal and perennial allergic rhinitis. The pump was designed with a shorter tip to avoid contact with the sensitive mucosal surfaces. New designs to reduce the need for priming and re-priming, and pumps incorporating pressure point features to improve the dose reproducibility and dose counters and lock-out mechanisms for enhanced dose control and safety are available (www.rexam.com and www.aptar.com).

Traditional, simple metered-dose spray pumps require priming and some degree of overfill to maintain dose conformity for the labeled number of doses. They are well suited for drugs to be administered daily over a prolonged duration, but due to the priming procedure and limited control of dosing, unless a specialty device is selected, they are less suited for drugs with a narrow therapeutic window, particularly if they are not used often. For expensive drugs and vaccines intended for single administration or sporadic use and where tight control of the dose and formulation is of particular importance, single-dose or bi-dose spray devices are preferred (www.aptar.com). A simple variant of a single-dose spray device (MAD™) is offered by LMA (LMA, Salt Lake City, UT, USA; www.lmana.com). A nosepiece with a spray tip is fitted to a standard syringe. The liquid drug to be delivered is first drawn into the syringe and then the spray tip is fitted onto the syringe. This device has been used in academic studies to deliver, for example, a topical steroid in patients with chronic rhinosinusitis and in a vaccine study. A pre-filled device based on the same principle for one or two doses (Accuspray™, Becton Dickinson Technologies, Research Triangle Park, NC, USA; www.bdpharma.com) is used to deliver the influenza vaccine FluMist (www.flumist.com), approved for both adults and children in the US market. A similar device for two doses was marketed by a Swiss company for delivery of another influenza vaccine a decade ago.

Pre-primed single- and bi-dose devices are also available, and consist of a reservoir, a piston, and a swirl chamber (see, e.g., the UDS UnitDose and BDS BiDose devices from Aptar, formerly Pfeiffer). The spray is formed when the liquid is forced out through the swirl chamber. These devices are held between the second and the third fingers with the thumb on the actuator. A pressure point mechanism incorporated in some devices secures reproducibility of the actuation force and emitted plume characteristics. Currently, marketed nasal migraine drugs like Imitrex® (www.gsk.com) and Zomig® (www.az.com; Pfeiffer/Aptar single-dose device), the marketed influenza vaccine Flu-Mist (www.flumist.com; Becton Dickinson single-dose spray device), and the intranasal formulation of naloxone for opioid overdose rescue, Narcan Nasal® (narcan.com; Adapt Pharma) are delivered with this type of device.

In certain embodiments, the 90% confidence interval for dose delivered per actuation is ±about 2%. In certain embodiments, the 95% confidence interval for dose delivered per actuation is ±about 2.5%.

Historically, intranasal administration of drugs in large volume, such as from syringes adapted with mucosal atomizer devices, has encountered difficulty due to the tendency of some of the formulation to drip back out of the nostril or down the nasopharynx. Accordingly, in certain embodiments, upon nasal delivery of said pharmaceutical composition to said patient, less than about 20% of said pharmaceutical composition leaves the nasal cavity via drainage into the nasopharynx or externally. In certain embodiments, upon nasal delivery of said pharmaceutical composition to said patient, less than about 10% of said pharmaceutical composition leaves the nasal cavity via drainage into the nasopharynx or externally. In certain embodiments, upon nasal delivery of said pharmaceutical composition to said patient, less than about 5% of said pharmaceutical composition leaves the nasal cavity via drainage into the nasopharynx or externally.

Current container closure system designs for inhalation spray drug products include both pre-metered and device-metered presentations using mechanical or power assistance and/or energy from patient inspiration for production of the spray plume. Pre-metered presentations contain previously measured doses or a dose fraction in some type of units (e.g., single or multiple blisters or other cavities) that are subsequently inserted into the device during manufacture or by the patient before use. Typical device-metered units have a reservoir containing formulation sufficient for multiple doses that are delivered as metered sprays by the device itself when activated by the patient.

A new nasal drug delivery method, which can be adapted to any type of dispersion technology for both liquids and powders, is breath-powered Bi-Directional™ technology. This concept exploits natural functional aspects of the upper airways to offer a delivery method that may overcome many of the inherent limitations of traditional nasal devices. Breath-powered Bi-Directional™ devices consist of a mouthpiece and a sealing nosepiece with an optimized frusto-conical shape and comfortable surface that mechanically expands the first part of the nasal valve. The user slides a sealing nosepiece into one nostril until it forms a seal with the flexible soft tissue of the nostril opening, at which point, it mechanically expands the narrow slit-shaped part of the nasal triangular valve. The user then exhales through an attached mouthpiece. When exhaling into the mouthpiece against the resistance of the device, the soft palate (or velum) is automatically elevated by the positive oropharyngeal pressure, isolating the nasal cavity from the rest of the respiratory system. This mechanism enables release of liquid or powder particles into an air stream that enters one nostril, passes entirely around the nasal septum, and exits through the opposite nostril.

With sterile filling, the use of preservatives is not required in pre-primed devices, but overfill is required resulting in a waste fraction similar to the metered-dose, multi-dose sprays. To emit 100 μL, a volume of 125 μL is filled in the device (Pfeiffer/Aptar single-dose device) used for the intranasal migraine medications Imitrex (sumatriptan) and Zomig (zolmitriptan) and about half of that for a bi-dose design. Sterile drug products may be produced using aseptic processing or terminal sterilization. Terminal sterilization usually involves filling and sealing product containers under high-quality environmental conditions. Products are filled and sealed in this type of environment to minimize the microbial and particulate content of the in-process product and to help ensure that the subsequent sterilization process is successful. In most cases, the product, container, and closure have low bioburden, but they are not sterile. The product in its final container is then subjected to a sterilization process such as heat or irradiation. In an aseptic process, the drug product, container, and closure are first subjected to sterilization methods separately, as appropriate, and then brought together. Because there is no process to sterilize the product in its final container, it is critical that containers be filled and sealed in an extremely high-quality environment. Aseptic processing involves more variables than terminal sterilization. Before aseptic assembly into a final product, the individual parts of the final product are generally subjected to various sterilization processes. For example, glass containers are subjected to dry heat; rubber closures are subjected to moist heat; and liquid dosage forms are subjected to filtration. Each of these manufacturing processes requires validation and control.

### Methods of Treatment

Provided herein are methods of treatment of alcohol use disorder and related conditions comprising the intranasal administration of a therapeutically effective amount of naltrexone or a salt or hydrate thereof.

### Sinclair Method

The Sinclair Method is a treatment for AUD that employs pharmacological extinction—the use of an opioid antagonist, such as naltrexone, to turn the habit-forming behavior of drinking alcohol into a habit-erasing behavior. The effect returns a person's craving for alcohol to its pre-addiction state.

The method consists of taking an oral dose of naltrexone about 1, about 2, about 3, or about 4 hours before a subject ingests alcohol. This pre-ingestion dose of oral naltrexone disrupts the body's behavior and reward cycle thereby causing the person to want to drink less instead of more. Most significantly, studies have shown that this methodology is equally effective with or without therapy, so subjects can choose whether or not to combine this treatment method with other therapies without negatively impacting the actual physical results. Importantly, unlike the other currently approved medication treatments for AUD, the Sinclair Method calls for the use of oral naltrexone while the individual continues their normal drinking behavior. As a result, maintenance of the medication treatment protocol is expected to be much higher than abstinence alone.

Using the Sinclair Method, extinction of AUD can occur within 6 months. However, the efficacy of oral naltrexone is hampered by slow onset, very low bioavailability and high levels of the peripherally selective active metabolite 6-β-naltrexol, and the injectable form of naltrexone presents itself with the obvious difficulties associated with needles including, for example, the need for administration by a practitioner at regularly scheduled intervals. Thus, intranasal administration of naltrexone, and use of absorption enhancers, in a pre-primed, single or multi-use nasal spray pump should significantly improve results in the treatment of AUD. An intranasal formulation of naltrexone absorbs quickly, providing fast onset of action and high bioavailability without the use of needles.

Accordingly, disclosed herein is a method of treatment of alcohol use disorder, or a related condition, in a subject, comprising administering to the subject an intranasal formulation comprising a therapeutically effective amount of naltrexone or a pharmaceutically acceptable salt thereof.

In certain embodiments, the intranasal formulation comprising naltrexone is administered prior to ingestion of alcohol.

In certain embodiments, the intranasal formulation comprising naltrexone is administered about 1 to about 2 hours prior to ingestion of alcohol. In certain embodiments, the intranasal formulation comprising naltrexone is administered about 1 hour prior to ingestion of alcohol. In certain embodiments, the intranasal formulation comprising naltrexone is administered about 0.5 to about 1 hours prior to ingestion of alcohol. In certain embodiments, the intranasal formulation comprising naltrexone is administered about 10 to about 30 minutes prior to ingestion of alcohol. In certain embodiments, the intranasal formulation comprising naltrexone is administered about 5 to about 10 minutes prior to ingestion of alcohol. In certain embodiments, the intranasal formulation comprising naltrexone is administered just before ingestion of alcohol.

In certain embodiments, the intranasal formulation comprising naltrexone is administered contemporaneously with the ingestion of alcohol.

In certain embodiments, the intranasal formulation comprising naltrexone is administered just after ingestion of alcohol. In certain embodiments, the intranasal formulation comprising naltrexone is administered within an hour after commencement of ingestion of alcohol.

It is expected that because intranasal naltrexone has a rapid uptake via the nasal mucosa and rapid appearance in the plasma, as evidenced by the studies below, intranasal administration will permit the subject to dose naltrexone much more immediately before, and even contemporaneously with or after, ingestion if alcohol, and experience benefits such as extinction, reduction in craving, etc. It is expected that absorption enhancers will further this effect.

In certain embodiments, the alcohol use disorder is alcohol abuse. In certain embodiments, the alcohol use disorder is alcohol dependence. In certain embodiments, the alcohol use disorder is alcoholism.

The methods disclosed herein may be achieved by administration of various embodiments of the formulations disclosed herein, for example above in the section “Pharmaceutical Formulations” and the Examples below. The formulations may be administered using devices known on the art, for example the devices disclosed herein in the section entitled “Nasal Drug Delivery Devices and Kits.”

For example, in certain embodiments, the intranasal formulation comprises an aqueous solution.

In certain embodiments, about 0.05-about 0.2 mL (about 50-about 200 μL) of said formulation is delivered to the subject per dose. In certain embodiments, about 0.1 mL (about 100 μL) of said formulation is delivered to the subject.

In certain embodiments, said formulation is at a concentration of about 40 mg/mL. In certain embodiments, said formulation is at a concentration of about 30 mg/mL. In certain embodiments, said formulation is at a concentration of about 20 mg/mL. In certain embodiments, said formulation is at a concentration of about 10 mg/mL. In certain embodiments, said formulation is at a concentration of about 5 mg/mL. In certain embodiments, said formulation is at a concentration of about 50, about 60, about 70, or about 80 mg/mL.

In certain embodiments, the intranasal formulation is administered as a single administration to one nostril. In certain embodiments, the intranasal formulation is administered as two administrations, one to each nostril.

In certain embodiments, the absorption enhancer is selected from the group consisting of benzalkonium chloride, chitosans, cyclodextrins, deoxycholic acid, dodecyl maltoside, glycocholic acid, laureth-9, taurocholic acid, and taurodihydrofusidic acid. In certain embodiments, the absorption enhancer is an alkylglycoside or alkylsaccharide. In certain embodiments, the alkylsaccharide is chosen from dodecyl maltoside, tetradecyl maltoside (TDM) and sucrose dodecanoate. In certain embodiments, the absorption enhancer is an alkylsaccharide. In certain embodiments, the alkylsaccharide is Intravail® (dodecyl maltoside).

In certain embodiments, each dose delivered comprises about 1, about 2, about 3, about 4, about 5, about 6, about 7, or about 8 mg naltrexone or a salt or hydrate thereof. In certain embodiments, each dose delivered comprises about 8 mg naltrexone or a salt or hydrate thereof. In certain embodiments, each dose delivered comprises about 4 mg naltrexone or a salt or hydrate thereof. In certain embodiments, each dose delivered comprises about 2 mg naltrexone or a salt or hydrate thereof. In certain embodiments, each dose delivered comprises about 1 mg naltrexone or a salt or hydrate thereof.

Also provided herein are embodiments wherein any embodiment described above may be combined with any one or more other embodiment(s), provided the combination is not mutually exclusive.

### EXAMPLES

The following examples are included to demonstrate preferred embodiments of the invention. The following examples are presented only by way of illustration and to assist one of ordinary skill in using the invention. The examples are not intended in any way to otherwise limit the scope of the invention. Those of skill in the art should, in light of the present disclosure, appreciate that many changes can be made in the specific embodiments which are disclosed and still obtain a like or similar result without departing from the spirit and scope of the invention.

### Example 1

### Intranasal Naltrexone Protocol for the Treatment of Alcohol Use Disorder

Individuals with alcohol use disorder (AUD) will be treated with intranasal naltrexone and examined for abstinence, reduced consumption of alcohol, and/or extinguished consumption of alcohol. Individuals with AUD are believed to release endogenous opioids upon the ingestion of alcohol. The binding of these opioids to receptors in the brain may be responsible for the positive reinforcing effects of alcohol. Drinking alcohol while the opioid antagonist naltrexone blocks the positive reinforcement from alcohol should extinguish alcohol drinking and craving.

In one example of a protocol, subjects (e.g., about 10-20) with AUD will make be admitted as in-patients to a study site. An initial visit serves the purpose of screening, to confirm the diagnosis and obtain informed consent. During their in-patient stay (e.g., one or more weeks), each subject will receive a placebo or intranasal dose of naltrexone followed by the consumption one or more alcoholic beverages. Naltrexone will be administered at the designated dose and by the designated method at about 0.25 to about 4 hrs before consumption of alcohol. One example of a dosing treatment is an intranasal formulation delivering about 1 to about 4 mg of naltrexone per administration, delivered by a single- or multi-use spray device. Another example of a dosing treatment is an intranasal formulation delivering a first dose of 4 mg of naltrexone in the morning, followed by subsequent doses of 4 mg of naltrexone throughout the day as needed by the patient. Yet another example of a dosing treatment is an intranasal formulation delivering up to 16 mg of naltrexone per day. The intranasal formulation of naltrexone may or may not contain an absorption enhancer, such as Intravail.

It is expected that intranasal naltrexone will improve post-treatment suppression of alcohol intake. It is also expected that intranasal naltrexone will reduce alcohol cravings and the amount of time required for a subject to exhibit pharmacological extinction of alcohol cravings.

Approximately 1 hour prior to dosing, ECG, blood pressure, heart rate, and respiration rate will be measured and the time will be recorded. At approximately 1 and 4 hours after dosing, the ECG will be repeated and the time will be recorded. Vital signs including sitting (after 5 minutes) heart rate, blood pressure and respiration rate will be measured pre-dose and approximately 1 and 4 hours after each dose. Adverse events (AEs) will be recorded and treatment terminated if necessary. The nasal passage will be examined at pre-dose, 5 minutes, 30 minutes, 60 minutes, 4 hours, and 24 hours post-dose after intranasal administration only.

At screening, admission, and discharge, ECG, and vital signs will be checked once per day. Vital signs will also be checked once on the day after naltrexone administration. AEs will be assessed by spontaneous reports by subjects, by examination of the nasal mucosa, by measuring vital signs, ECG, and clinical laboratory parameters.

### Example 2

### Pharmacokinetic Data Analysis

The non-compartmental pharmacokinetic (PK) parameters of naltrexone and 6β-naltrexol (Cₘₐₓ, Tₘₐₓ, AUC₀₋ₜ, AUC_{0-∞}, t_{1/2}, λz, and apparent clearance (CL/F, naltrexone only) will be determined. PK parameters of various AUD treatment protocols (e.g., 4 mg intranasal with or without an absorption enhancer such as an alkylsacchraide; 50 mg oral tablet) will be compared with a 2 mg intramuscular (IM) dose of naltrexone. Dose-adjusted values for AUCs and Cₘₐₓ will be calculated. The relative extent of intranasal (IN) and oral absorption (PO) absorption will be estimated from the dose-corrected AUCs. Within an ANOVA framework, comparisons of IN-transformed PK parameters for IN and PO versus IM naltrexone treatments will be performed. The 90% confidence interval for the ratio (IN/IM and PO/IM) of the geometric least squares means of AUC and Cₘₐₓ parameters will be constructed for comparison of each treatment with IM naltrexone. These 90% confidence intervals will be obtained by exponentiation of the 90% confidence intervals for the difference between the least squares means based upon a log scale.

AEs will be coded using the most recent version of the Medical Dictionary for Regulatory Activities (MedDRA) preferred terms and will be grouped by system, organ, class (SOC) designation. The severity, frequency, and relationship of AEs to study drug will be presented by preferred term by SOC grouping. Separate summaries will be provided for the 4 study periods: after the administration of each dose of study drug up until the time of the next dose of study drug or clinic discharge. Listings of each individual AE including start date, stop date, severity, relationship, outcome, and duration will be provided.

Clinically significant changes in vital signs, ECG, and clinical laboratory parameters will be presented as counts and percentages by dosing session.

### Example 3. Dose Selection and Pharmaceutical Composition with Absorption Enhancers

Intranasal naltrexone may optionally be formulated with absorption-enhancing excipients.

One such excipient is the alkylsaccharide Intravail®. Concentrations of Intravail® in nasal formulations have generally been 0.1% and 0.2% by weight. The present study will use a concentration of 0.25% by weight of an alkylsacchraide. Concentrations of 25% Intravail® were non-irritating in the rabbit eye model. The oral “no observable effect level” was approximately 20,000 to 30,000 mg/kg body weight. While there is no comparable intranasal data, the essential lack of oral safety suggests that the amount of an alkylsacchraide needed for nasal toxicity would be much higher than the amount that will be administered in this study.

In the present study, a single dose of naltrexone was administered 4 ways: a) 4 mg IN in sterile water for injection; b) 4 mg IN in sterile water for injection with 0.25% Intravail®; c) 2 mg as an IM injection; and d) a 50-mg oral tablet. Intranasal administration is expected to increase the rate of absorption as compared to oral administration. Addition of Intravail® is expected to further increase the rate of absorption from the nasal passages.

### Example 4. Pharmacokinetic Evaluation of Intranasal Naltrexone

### Study Goals.

The purpose of this clinical study was twofold: to determine the pharmacokinetics of two intranasal formulations (4 mg with and without Intravail) and one oral formulation (50 mg tablet) of naltrexone compared to a 2-mg intramuscular dose of naltrexone; and to determine the safety of intranasal naltrexone, particularly with respect to nasal irritation, such as inflammation (erythema, edema, and erosion) and bleeding. To that end, the study's primary endpoints were the pharmacokinetic parameters (Cₘₐₓ, Tₘₐₓ, AUC₀₋ₜ, and AUC_{0-inf}) of the IN and oral naltrexone formulations compared with an IM dose of 2 mg of naltrexone. Secondary endpoints included adverse events (AEs), vital signs (heart rate, sitting blood pressure, and respiration rate), electrocardiogram (ECG), clinical laboratory changes and nasal irritation using the nasal irritation scale.

### Study Design.

Fourteen healthy volunteers were enrolled and completed all study drug administrations and blood collections for PK assessments. This was an in-patient open-label, crossover study involving approximately 14 healthy volunteers. Each subject received each naltrexone treatment: 4 mg IN (one 0.1 mL spray of a 40 mg/mL solution in one nostril), 4 mg plus Intravail IN (one 0.1 mL spray of a 40 mg/mL solution containing 0.25% Intravail in one nostril), 2 mg IM, and 50 mg oral tablet. Subjects stayed in the in-patient facility for 13 days to complete the entire study. Subjects were called 3 to 5 days after discharge to inquire concerning AEs and concomitant medications since discharge. Informed consent was obtained from all subjects, and all were screened for eligibility to participate in the study including medical history, physical examination, clinical chemistry, coagulation markers, hematology, infectious disease serology, urinalysis, urine drug and alcohol toxicology screen, vital signs and ECG.

On the day after clinic admission, subjects were administered study drug with a 3-day washout period between doses until all treatments had been administered. Blood was collected for analysis prior to dosing and approximately 2.5, 5, 10, 15, 20, 30, 45, 60 minutes and 2, 3, 4, 6, 8, 12, 16, 24, 30, 36, and 48 hours after study drug administration. On days of study drug administration, a 12-lead ECG was performed approximately 1 hour prior to dosing and at approximately 1 and 4 hours post-dose. Vital signs were measured pre-dose and approximately 1 and 4 hours post-dose.

On dosing days, the order of assessments were ECG, vital signs, then PK blood collection when scheduled at the same nominal times. The target time of the PK blood collection was considered the most critical and if the collection was more than ±1 minute from the scheduled time for the first 60 minutes of collections or more than ±5 minutes for the scheduled time points thereafter, this was considered a protocol deviation. ECG and vital signs were collected within the 10 minute period before the nominal time of blood collections. At screening, admission, and discharge, ECG, and vital signs were checked once per day. Vital signs were also checked once on the day after naltrexone administration. Clinical laboratory measurements were repeated after the last PK blood draw prior to clinic discharge. AEs were assessed by spontaneous reports by subjects, by examination of the nasal mucosa, by measuring vital signs, ECG, and clinical laboratory parameters.

### Inclusion and Exclusion Criteria:

1. Males and females 18 to 55 years of age, inclusive were included in this study. Written informed consent was required. Subject had to:
have body mass index (BMI) ranging from 18 to 30 kg/m², inclusive;
have adequate venous access;
have no clinically significant concurrent medical conditions determined by medical history, physical examination, clinical laboratory examination, vital signs, and 12-lead ECG;
agree to use an acceptable method of contraception, other than oral contraceptives, throughout the study and for 90 days after the last study drug administration (30 days for women); and
agree not to ingest alcohol, drinks containing xanthine >500 mg/day (e.g., Coca-Cola®, tea, coffee, etc.), or grapefruit/grapefruit juice or participate in strenuous exercise 72 hours prior to admission through the last blood draw of the study.

Exclusion criteria included:
any IN conditions including abnormal nasal anatomy, nasal symptoms (i.e., blocked and/or runny nose, nasal polyps, etc.);
having a product sprayed into the nasal cavity prior to screening and drug administration;
having been administered an investigational drug within 30 days prior to Day-1;
having taken prescribed or over-the-counter medications, dietary supplements, herbal products, vitamins, or recent use of opioid analgesics for pain relief (within 14 days of last use of any of these products);
a positive urine drug test for alcohol, opioids, cocaine, amphetamine, methamphetamine, benzodiazepines, tetrahydrocannabinol (THC), barbiturates, or methadone at screening or admission;
previous or current opioid, alcohol, or other drug dependence (excluding nicotine and caffeine), based on medical history;
consumption of greater than 20 cigarettes per day on average, in the month prior to screening, or would be unable to abstain from smoking (or use of any nicotine-containing substance) for at least one hour prior to and 2 hours after naltrexone dosing;
systolic blood pressure less than 90 mm Hg or greater than 140 mm Hg; diastolic blood pressure less than 55 mmHg or greater than 90 mmHg; respiratory rate less than 8 respirations per minute or greater than 20 respirations per minute;
on standard 12-lead ECG, a QTcF interval >440 msec for males and >450 msec for females; significant acute or chronic medical disease (investigator judgment);
a likely need for concomitant medication treatment during the study;
donated or received blood or underwent plasma or platelet apheresis within the 60 days prior to Day-1;
female who is pregnant, breast feeding, or plans to become pregnant during the study period or within 30 days after the last naltrexone administration;
positive test for hepatitis B surface antigen (HBsAg), hepatitis C virus antibody (HCVAb) or human immunodeficiency virus antibody (HIVAb) at screening;
current or recent (within 7 days prior to screening) upper respiratory tract infection; and
abnormal liver function test (ALT, AST, total bilirubin)>1.5 times upper limit of normal

### Study Drugs and Dosing.

Naltrexone hydrochloride (HCl) was obtained from Mallinckrodt Pharmaceuticals. The IN (40 mg/mL) formulations were made by the staff pharmacist at Vince & Associates; the vehicle for the IN formulations was sterile water for injection. The IM formulation (2 mg/mL) was made by the staff pharmacist at Vince & Associates; the vehicle was sterile saline for injection. IN naltrexone was administered using an Aptar multi-dose device with the subject in a reclined position (approximately 45 degrees). The subject was instructed not to breathe through the nose when the IN dose of naltrexone was administered. Naltrexone HCl for the IM injection was administered with a 23-g needle as a single 1-mL injection into the gluteus maximus muscle. Naltrexone HCl for oral administration (50 mg tablet) was sourced from a commercial supplier and administered with 240 mL water.

Naltrexone was administered on Days 1, 4, 7, and 10, in the following order: 4 mg naltrexone IN, 4 mg naltrexone plus Intravail® IN, 2 mg IM, and 50 mg oral. Subjects stayed in the in-patient facility for 13 days to complete the entire study and were discharged 2 days after the fourth dose.

### PK Assessments.

Blood (4 mL) was collected in sodium heparin containing tubes for PK analysis prior to dosing and 2.5, 5, 10, 1.5, 20, 30, 45, 60 minutes and 2, 3, 4, 6, 8, 12, 16, 24, 30, 36, and 48 hours after the start of study drug administration. Plasma was separated from whole blood and stored frozen at ≤20° C. until assayed. Naltrexone and 6β-naltrexol plasma concentrations were determined by liquid chromatography with tandem mass spectrometry at XenoBiotic laboratories, Inc., Plainsboro, New Jersey.

### Safety Assessments.

Heart rate, blood pressure, and respiration rate were recorded approximately 1 hour before naltrexone dosing and approximately 1 and 4 hours after dosing. A 12-lead ECG was obtained approximately 1 hour before and 1 and 4 hours after each naltrexone dose. ECG and vital signs was performed within the 10 minute period before the nominal time for post-dose blood collections. AEs were recorded from the start of study drug administration until clinic discharge. AEs were recorded relative to each dosing session to attempt to establish a relationship between the AE and type of naltrexone dose administered. An examination of the nasal passage was conducted at Day-1 to establish eligibility and at pre-dose, 5 minutes, 30 minutes, 60 minutes, 4 hours, and 24 hours post IN naltrexone administration to evaluate evidence of irritation to the nasal mucosa after IN administration only.

### Analysis.

Non-compartmental PK parameters of naltrexone and 6β-naltrexol, including, Tₘₐₓ, AUC₀₋ₜ, and AUC_{0-inf}, t_{1/2}, λ_{z}, and apparent clearance (CL/F, naltrexone only), was determined. Pharmacokinetic parameters (Cₘₐₓ, Tₘₐₓ and AUCs) for IN and PO naltrexone were compared with those for IM naltrexone. Dose-adjusted values for AUCs and Cₘₐₓ were calculated. The relative extent of IN and PO absorption (IN and PO versus IM) will be estimated from the dose-corrected AUCs. Within an ANOVA framework, comparisons of In-transformed PK parameters (Cₘₐₓ and AUC) for IN and PO versus IM naltrexone treatments were performed. The 90% confidence interval for the ratio (IN/IM and PO/IM) of the geometric least squares means of AUC and Cₘₐₓ parameters were constructed for comparison of each treatment with IM naltrexone. These 90% CIs were obtained by exponentiation of the 90% CIs for the difference between the least squares means based upon an In scale.

### Results.

Results are shown below in Tables 1-5.

TABLE 1
Mean (SD) concentrations of naltrexone following a
single IN, IM or oral administration to healthy subjects.

| | Treatment | | | | | | | |
|---|---|---|---|---|---|---|---|---|
| | | | 4 mg IN + | | | | | |
| Hour | 4 mg IN | | 0.25% Intravail | | 2 mg IN | | 50 mg Oral | |
| 0 | 0 | 0 | 0 | 0 | 0 | 0 | 0 | 0 |
| 0.042 | 0.117 | (0.17) | 1.15 | (0.919) | 0.678 | (1.69) | 0 | 0 |
| 0.083 | 1.51 | (1.62) | 11.9 | (9.69) | 1.04 | (1.26) | 0.109 | (0.232) |
| 0.17 | 3.4 | (3.86) | 12.1 | (6.36) | 2.97 | (2) | 0.851 | (1.5) |
| 0.25 | 4.36 | (3.71) | 10.4 | (3.93) | 3.45 | (1.58) | 2.5 | (3.54) |
| 0.33 | 4.46 | (3.62) | 9.81 | (2.41) | 3.58 | (1.46) | 4.75 | (4.71) |
| 0.5 | 4.08 | (1.99) | 7.19 | (2.08) | 3.43 | (1.06) | 7.16 | (4.69) |
| 0.75 | 3.39 | (1.46) | 5.46 | (1.48) | 3.02 | (0.749) | 6.9 | (3.54) |
| 1 | 3.19 | (1.51) | 4.55 | (1.78) | 2.73 | (0.676) | 6.34 | (2.78) |
| 2 | 2.33 | (0.832) | 3.07 | (1.27) | 2.35 | (0.698) | 5.22 | (1.73) |
| 3 | 1.5 | (0.633) | 2 | (0.885) | 1.79 | (0.491) | 3.66 | (1.76) |
| 4 | 1.02 | (0.369) | 1.25 | (0.465) | 1.3 | (0.341) | 2.39 | (1.16) |
| 6 | 0.418 | (0.193) | 0.536 | (0.188) | 0.584 | (0.185) | 1.13 | (0.462) |
| 8 | 0.22 | (0.0941) | 0.267 | (0.105) | 0.242 | (0.0803) | 0.596 | (0.426) |
| 12 | 0.0641 | (0.021) | 0.0726 | (0.028) | 0.0626 | (0.0269) | 0.3 | (0.183) |
| 16 | 0.0214 | (0.0131) | 0.0226 | (0.0165) | 0.0101 | (0.0132) | 0.141 | (0.104) |
| 24 | 0.00462 | (0.0117) | 0 | 0 | 0 | 0 | 0.0657 | (0.0528) |
| 30 | 0.00187 | (0.00674) | 0 | 0 | 0 | 0 | 0.0345 | (0.0224) |
| 36 | 0 | 0 | 0 | 0 | 0 | 0 | 0.0207 | (0.0255) |
| 48 | 0.0018 | (0.0065) | 0 | 0 | 0 | 0 | 0 | 0 |

TABLE 2
Mean CV %) PK Parameters for Naltrexone Following Administration to Healthy Subjects

| | | | 4 mg IN plus | | | | | |
|---|---|---|---|---|---|---|---|---|
| PK Parameter | 4 mg IN^{a} | | 0.25% Intravail ^{b} | | 2 mg IM^{c} | | 50 mg Oral^{c} | |
| Cₘₐₓ (ng/mL) | 5.35 | (66.8) | 15.7 | (52.0) | 4.10 | (34.0) | 9.34 | (31.8) |
| Cₘₐₓ/Dose (ng/mL/mg) | 1.48 | (66.8) | 4.35 | (52.0) | 2.27 | (34.0) | 0.206 | (31.8) |
| Tₘₐₓ (h) ^{d} | 0.50 | (0.17, 2.00) | 0.17 | (0.083, 0.33) | 0.33 | (0.17, 1.00) | 0.50 | (0.33, 3.00) |
| AUC₀₋ₜ (h · ng/mL) | 11.9 | (34.1) | 18.3 | (31.2) | 12.1 | (25.5) | 26.5 | (32.3) |
| AUC₀₋ₜ/Dose (h · ng/mL/mg) | 3.28 | (34.1) | 5.07 | (31.2) | 6.71 | (25.5) | 0.587 | (32.3) |
| AUC_{0-inf} (h · ng/mL) | 12.0 | (33.7) | 18.5 | (31.0) | 12.3 | (25.6) | 26.9 | (31.8) |
| AUC_{0-inf}/Dose (h · ng/mL/mg) | 3.32 | (33.7) | 5.10 | (31.0) | 6.78 | (25.6) | 0.594 | (31.8) |
| AUCₑₓₜᵣₐₚ (%) | 1.09 | (57.0) | 0.707 | (44.0) | 1.01 | (71.7) | 1.38 | (70.1) |
| CL/F (L/h) | 330 | (28.9) | 214 | (33.6 | 154 | (19.0) | 1890 | (41.4) |
| λ_{z} (1/h) | 0.281 | (15.1) | 0.317 | (15.1) | 0.361 | (16.8) | 0.122 | (38.0) |
| t_{1/2} (h) | 2.52 | (14.9) | 2.23 | (14.9) | 1.97 | (15.5) | 6.41 | (36.6) |

| | | | | | | | |
|---|---|---|---|---|---|---|---|
| Fᵣₑₗ | 0.481 | (36.1)c | 0.783 | (17.7)^{c} | NA | 0.0903 | (37.0) |

NA = Not applicable;
Frel = Bioavailability relative to IM dose, calculated as ratio of AUCinf/Dose for IN or PO route relative to IM route.
^{a}N = 13;
^{b} N = 12;
^{c}N = 10;
^{d} Median (minimum, maximum)

Following IN administration of 4 mg naltrexone, the mean concentration at 2.5 minutes postdose was 0.117 ng/mL. When 0.25% Intravaile was added to the formulation, the mean concentration was 10 times greater (1.15 ng/mL) at 2.5 minutes. At 5 minutes postdose, the mean concentrations of naltrexone with and without Intravail® were 11.9 ng/ml and 1.51 ng/mL, respectively, an 8-fold difference. The addition of 0.25% Intravaile to the IN formulation decreased median Tₘₐₓ from 30 minutes to 10 minutes and increased Cₘₐₓ almost 3-fold (15.7 versus 5.35 ng/mL). Overall exposure as measured by AUC_{0-inf} increased by 54%, indicating that the main effect of Intravail® was to increase the rate of absorption more than the extent.

The mean plasma concentrations of naltrexone at 2.5 and 5 minutes after administration of 2 mg naltrexone IM were 0.678 ng/mL and 1.04 ng/ml, respectively. The mean Cₘₐₓ value of 4.10 ng/mL 20 minutes after the 2 mg IM dose was 23% less than after the 4 mg IN dose and 74% less compared to when Intravail® was part of the IN formulation.

The mean Cₘₐₓ value after the oral dose was 9.34 ng/mL, which was less than observed after the IN dose with Intravail even though 50 mg was administered orally compared to only 4 mg IN.

The mean terminal phase half-life (t_{1/2}) of naltrexone was 1.97 hours to 2.52 hours after IM and IN administration. The t_{1/2} was 6.41 hours after the oral dose.

When AUC_{0-inf} values were corrected for dose, the relative bioavailability of naltrexone after the IN doses with and without 0.25% Intravail® was 78% and 48%, respectively, compared to the IM administration. The relative bioavailability for the oral dose was only 9%, indicating extensive first pass metabolism by the gastrointestinal tract and liver.

Statistical analysis of dose-adjusted PK parameters suggested exposure for the IN dose was approximately 48% or 60% of the IM dose on a per mg basis, in terms of geometric least-squares mean (GM) dose-adjusted AUC and Cₘₐₓ, respectively. IN administration of naltrexone with 0.25% Intravail® resulted in dose-adjusted exposure that was higher than the IM route in terms of Cₘₐₓ (geometric least-squares mean ratio between treatments [GMR] of 188%) and lower in terms of AUC (GMR of 76%). For the oral route, the GMR for dose-adjusted naltrexone exposure was approximately 9% of the IM dose.

TABLE 3
Mixed-Effects ANOVA Results for Naltrexone Pharmacokinetic
Parameters Following Intranasal or Oral Administration
vs. Intramuscular Administration to Healthy Subjects

| | Comparison | | 90% CI of GMR (%) | |
|---|---|---|---|---|
| PK Parameter | (2 mg IM Reference) | GMR (%) | Lower | Upper |
| Cmax (ng/mL) | 4 mg IN vs 2 mg IM | 121 | 91.1 | 160 |
| | 4 mg IN plus Intravail vs 2 mg IM | 377 | 321 | 442 |
| | 50 mg PO vs 2 mg IM | 231 | 190 | 282 |
| AUC0-t | 4 mg IN vs 2 mg IM | 96.4 | 82.6 | 112 |
| (h · ng/mL) | 4 mg IN plus Intravail vs 2 mg IM | 152 | 136 | 169 |
| | 50 mg PO vs 2 mg IM | 221 | 182 | 268 |
| AUC0-inf | 4 mg IN vs 2 mg IM | 96.4 | 82.7 | 112 |
| (h · ng/mL) | 4 mg IN plus Intravail vs 2 mg IM | 151 | 136 | 168 |
| | 50 mg PO vs 2 mg IM | 221 | 183 | 268 |
| Cmax/Dose | 4 mg IN vs 2 mg IM | 60.4 | 45.5 | 80.2 |
| (ng/mL/mg) | 4 mg IN plus Intravail vs 2 mg IM | 188 | 161 | 221 |
| | 50 mg PO vs 2 mg IM | 9.3 | 7.6 | 11.3 |
| AUC0-t /Dose | 4 mg IN vs 2 mg IM | 48.2 | 41.3 | 56.2 |
| (h · ng/mL/mg) | 4 mg IN plus Intravail vs 2 mg IM | 75.9 | 68.1 | 84.6 |
| | 50 mg PO vs 2 mg IM | 8.8 | 7.3 | 10.7 |
| AUC0-inf/Dose | 4 mg IN vs 2 mg IM | 48.2 | 41.4 | 56.2 |
| (h · ng/mL/mg) | 4 mg IN plus Intravail vs 2 mg IM | 75.7 | 68 | 84.2 |
| | 50 mg PO vs 2 mg IM | 8.9 | 7.3 | 10.7 |

GMR = Geometric least-squares mean ratio between treatments (expressed as percentage of reference)

The mean Cₘₐₓ values of 6β-naltrexol were 1.5 ng/ml after the IM administration and approximately 3 ng/ml after the IN administration; Cₘₐₓ was 90.7 ng/ml after the 50 mg oral dose (Table 2-3). When adjusted for the administered dose, the Cₘₐₓ values were similar for the IN and IM doses (0.833 and 0.838 ng/ml/mg) but approximately 2-fold higher (2.00 ng/mL/mg) after oral administration.

Values of AUCO-inf also were increased considerably after the oral dose in comparison to the IN and IM doses (675 h·ng/ml and 44.0 to 27.1 h·ng/mL, respectively). The greater extent of first pass metabolism of naltrexone was evident in the ratio of AUCO-inf for 6β-naltrexol compared to that of naltrexone: after the IN and IM doses, the ratio was approximately 2.2 to 3.7 while it was 25 after the oral dose.

The mean t_{1/2} of the metabolite was 12.4 to 13.9 hours and was independent of the route of administration.

TABLE 4
Mean (SD) concentrations of 6β-naltrexol following a
single IN, IM or oral administration to healthy subjects.

| | Treatment | | | | | | | |
|---|---|---|---|---|---|---|---|---|
| | | | 4 mg IN + | | | | | |
| Hour | 4 mg IN | | 0.25% Intravail | | 2 mg IN | | 50 mg Oral | |
| 0 | 0 | 0 | 0.0682 | (0.0257) | 0.0661 | (0.0256) | 0.0454 | (0.0141) |
| 0.042 | 0 | 0 | 0.082 | (0.0378) | 0.0627 | (0.0248) | 0.0448 | (0.0202) |
| 0.083 | 0.0321 | (0.0432) | 0.238 | (0.146) | 0.12 | (0.117) | 1.4 | (3.49) |
| 0.17 | 0.196 | (0.196) | 0.994 | (0.558) | 0.283 | (0.281) | 14.2 | (31) |
| 0.25 | 0.45 | (0.448) | 1.86 | (0.763) | 0.454 | (0.293) | 31.6 | (47.3) |
| 0.33 | 0.693 | (0.624) | 2.55 | (0.918) | 0.677 | (0.385) | 45.5 | (43.7) |
| 0.5 | 1.11 | (0.559) | 2.84 | (0.748) | 0.852 | (0.328) | 68.7 | (41.3) |
| 0.75 | 1.82 | (1.16) | 2.93 | (0.757) | 1.08 | (0.452) | 60.8 | (28.2) |
| 1 | 1.83 | (0.815) | 2.73 | (0.481) | 1.1 | (0.404) | 58.6 | (18.2) |
| 2 | 2.68 | (0.842) | 2.9 | (0.767) | 1.39 | (0.462) | 54 | (16.3) |
| 3 | 2.61 | (0.793) | 2.61 | (0.708) | 1.48 | (0.402) | 45.8 | (15.2) |
| 4 | 2.37 | (0.669) | 2.45 | (0.598) | 1.46 | (0.388) | 38 | (12.2) |
| 6 | 1.97 | (0.554) | 2.03 | (0.399) | 1.3 | (0.252) | 28.5 | (7.52) |
| 8 | 1.62 | (0.418) | 1.67 | (0.27) | 1.08 | (0.165) | 22.2 | (5.51) |
| 12 | 1.26 | (0.299) | 1.25 | (0.176) | 0.81 | (0.131) | 15.4 | (2.85) |
| 16 | 0.919 | (0.229) | 0.923 | (0.155) | 0.595 | (0.115) | 11.4 | (1.78) |
| 24 | 0.602 | (0.194) | 0.618 | (0.145) | 0.365 | (0.0808) | 8.14 | (2.09) |
| 30 | 0.418 | (0.114) | 0.457 | (0.116) | 0.255 | (0.0666) | 5.93 | (1.85) |
| 36 | 0.292 | (0.0868) | 0.312 | (0.0862) | 0.18 | (0.0478) | 4.35 | (1.54) |
| 48 | 0.184 | (0.0645) | 0.175 | (0.0623) | 0.106 | (0.0329) | 2.43 | (0.882) |

TABLE 5
Mean (CV %) PK Parameters for 6β-Naltrexol Following Administration to Healthy Subjects

| | | | 4 mg IN plus | | | | | |
|---|---|---|---|---|---|---|---|---|
| PK Parameter | 4 mg IN^{a} | | 0.25% Intravail ^{b} | | 2 mg IM^{c} | | 50 mg Oral^{c} | |
| Cₘₐₓ (ng/mL) | 3.01 | (33.2) | 3.29 | (23.7) | 1.52 | (26.8) | 90.7 | (30.3) |
| Cₘₐₓ/Dose (ng/mL/mg) | 0.833 | (33.2) | 0.908 | (23.7) | 0.838 | (26.8) | 2.00 | (30.3) |
| Tₘₐₓ (h) ^{d} | 2.00 | (0.75, 6.0) | 0.75 | (0.25, 4.00) | 3.00 | (0.75, 4.00) | 0.63 | (0.25, 3.00) |
| AUC₀₋ₜ (h · ng/mL) | 40.3 | (23.3) | 43.0 | (17.4) | 25.1 | (18.3) | 614 | (19.5) |
| AUC₀₋ₜ/Dose | 11.1 | (23.3) | 11.9 | (17.4) | 13.9 | (18.3) | 13.6 | (19.5) |
| (h · ng/mL/mg) | | | | | | | | |
| AUC_{0-inf} (h · ng/mL) | 44.0 | (23.1) | 46.3 | (18.3) | 27.1 | (19.0) | 675 | (19.9) |
| AUC_{0-inf}/Dose | 12.2 | (23.1) | 12.8 | (18.3) | 15.0 | (19.0) | 14.9 | (19.9) |
| (h · ng/mL/mg) | | | | | | | | |
| AUCₑₓₜᵣₐₚ (%) | 8.57 | (46.1) | 7.02 | (37.3) | 7.02 | (29.4) | 8.79 | (57.3) |
| λ_{z} (1/h) | 0.0530 | (21.8) | 0.0553 | (15.1) | 0.0570 | (14.8) | 0.0510 | (15.8) |
| t_{1/2} (h) | 13.7 | (22.7) | 12.8 | (14.6) | 12.4 | (13.2) | 13.9 | (15.9) |

*Median (Min, Max) statistics presented for Tmax.
All other values presented as: Mean (Percent coefficient of variation);
^{a}N = 13;
^{b} N = 12;
^{c}N = 10;
^{d} Median (minimum, maximum)

With the exception of the mean Cₘₐₓ of naltrexone following the 4 mg IN dose, which was approximately 2-fold higher in females compared to males, there was no clinically meaningful difference between the sexes for the PK parameters of either naltrexone or 6β-naltrexol following IN, IM, or PO administration.

### Safety.

In total, 10 of 14 subjects (71%) in the safety population experienced at least one AE (any dosing period, any relationship to drug). The most frequent AEs were of the Nervous System Disorders SOC (7 subjects, 50%), and dizziness was the most frequent AE regardless of severity or attribution (5 subjects, 36%). No severe AEs were observed, and only one moderate AE was observed, a case of dizziness after the first dose (4 mg IN) that was considered related to the study agent. Three subjects experienced AEs that were unexpected (UAE, defined as AEs that were not described with respect to nature, severity, or frequency in the current product package insert): two UAEs were considered unrelated to the study agent and one treatment-related UAE of mild syncope after administration of the Day 1 dose (4 mg IN). Three subjects were discontinued from the study due to AEs (hypertension, syncope, and out-of-range pre-dose vital signs).

### Example 5. Formulations of Intranasal Naltrexone

The following tables set forth examples of formulations of naltrexone for intranasal administration for the treatment of disorders such as Alcohol Use Disorder. Table 6 sets forth simple aqueous solution formulations such as those used in the experiment above, to be dispensed in increments of about 100 μL.

TABLE 6

| | Naltrexone | | | |
|---|---|---|---|---|
| | HCl, dose | Absorption | μL per | Conc., |
| Ex. | (mg) | Enhancer | dose | mg/mL |
| 1 | 1 | n/a | 100 | 10 |
| 2 | 1 | Intravail 0.25% | 100 | 10 |
| 3 | 2 | n/a | 100 | 20 |
| 4 | 2 | Intravail 0.25% | 100 | 20 |
| 5 | 3 | n/a | 100 | 30 |
| 6 | 3 | Intravail 0.25% | 100 | 30 |
| 7 | 4 | n/a | 100 | 40 |
| 8 | 4 | Intravail 0.25% | 100 | 40 |
| 9 | 5 | n/a | 100 | 50 |
| 10 | 5 | Intravail 0.25% | 100 | 50 |
| 11 | 6 | n/a | 100 | 60 |
| 12 | 6 | Intravail 0.25% | 100 | 60 |
| 13 | 7 | n/a | 100 | 70 |
| 14 | 7 | Intravail 0.25% | 100 | 70 |
| 15 | 8 | n/a | 100 | 80 |
| 16 | 8 | Intravail 0.25% | 100 | 80 |

Table 7 sets forth formulations for intranasal administration in 100 μL of an aqueous solution including excipients such as an isotonicity agent, a stabilizing agent, and/or a compound which acts as a preservative or surfactant. EDTA stands for disodium edetate and BZK stands for benzalkonium chloride.

TABLE 7

| | Naltrexone | Absorption | Isotonicity | Stabilizing | Preservative/ |
|---|---|---|---|---|---|
| Ex. | HCl | Enhancer | Agent | Agent | Surfactant |
| 17 | 2 mg | n/a | NaCl 0.74% | n/a | n/a |
| 18 | 2 mg | n/a | NaCl 0.74% | EDTA 0.2% | n/a |
| 19 | 2 mg | n/a | NaCl 0.74% | n/a | BZK 0.01% |
| 20 | 2 mg | n/a | NaCl 0.74% | EDTA 0.2% | BZK 0.01% |
| 21 | 4 mg | n/a | NaCl 0.74% | n/a | n/a |
| 22 | 4 mg | n/a | NaCl 0.74% | EDTA 0.2% | n/a |
| 23 | 4 mg | n/a | NaCl 0.74% | n/a | BZK 0.01% |
| 24 | 4 mg | n/a | NaCl 0.74% | EDTA 0.2% | BZK 0.01% |
| 25 | 2 mg | Intravail 0.25% | NaCl 0.74% | n/a | n/a |
| 26 | 2 mg | Intravail 0.25% | NaCl 0.74% | EDTA 0.2% | n/a |
| 27 | 2 mg | Intravail 0.25% | NaCl 0.74% | n/a | BZK 0.01% |
| 28 | 2 mg | Intravail 0.25% | NaCl 0.74% | EDTA 0.2% | BZK 0.01% |
| 29 | 4 mg | Intravail 0.25% | NaCl 0.74% | n/a | n/a |
| 30 | 4 mg | Intravail 0.25% | NaCl 0.74% | EDTA 0.2% | n/a |
| 31 | 4 mg | Intravail 0.25% | NaCl 0.74% | n/a | BZK 0.01% |
| 32 | 4 mg | Intravail 0.25% | NaCl 0.74% | EDTA 0.2% | BZK 0.01% |
| 33 | 2 mg | Intravail 0.18% | NaCl 0.74% | n/a | n/a |
| 34 | 2 mg | Intravail 0.18% | NaCl 0.74% | EDTA 0.2% | n/a |
| 35 | 2 mg | Intravail 0.18% | NaCl 0.74% | n/a | BZK 0.01% |
| 36 | 2 mg | Intravail 0.18% | NaCl 0.74% | EDTA 0.2% | BZK 0.01% |
| 37 | 4 mg | Intravail 0.18% | NaCl 0.74% | n/a | n/a |
| 38 | 4 mg | Intravail 0.18% | NaCl 0.74% | EDTA 0.2% | n/a |
| 39 | 4 mg | Intravail 0.18% | NaCl 0.74% | n/a | BZK 0.01% |
| 40 | 4 mg | Intravail 0.18% | NaCl 0.74% | EDTA 0.2% | BZK 0.01% |
| 41 | 2 mg | Benzalkonium | NaCl 0.74% | n/a | n/a |
| | | chloride, 0.01% | | | |
| 42 | 2 mg | Benzalkonium | NaCl 0.74% | EDTA 0.2% | n/a |
| | | chloride, 0.01% | | | |
| 43 | 2 mg | Benzalkonium | NaCl 0.74% | n/a | BZK 0.01% |
| | | chloride, 0.01% | | | |
| 44 | 2 mg | Benzalkonium | NaCl 0.74% | EDTA 0.2% | BZK 0.01% |
| | | chloride, 0.01% | | | |
| 45 | 4 mg | Benzalkonium | NaCl 0.74% | n/a | n/a |
| | | chloride, 0.01% | | | |
| 46 | 4 mg | Benzalkonium | NaCl 0.74% | EDTA 0.2% | n/a |
| | | chloride, 0.01% | | | |
| 47 | 4 mg | Benzalkonium | NaCl 0.74% | n/a | BZK 0.01% |
| | | chloride, 0.01% | | | |
| 48 | 4 mg | Benzalkonium | NaCl 0.74% | EDTA 0.2% | BZK 0.01% |
| | | chloride, 0.01% | | | |

Also provided are examples 1-48A which additionally contain an amount of hydrochloric acid sufficient to achieve a pH of 3.5-5.5. The acid should be pharmaceutically acceptable, for example, hydrochloric acid.

### OTHER EMBODIMENTS

The detailed description set forth herein is provided to aid those skilled in the art in practicing the present disclosure. However, the disclosure described and claimed herein is not to be limited in scope by the specific embodiments herein disclosed because these embodiments are intended as illustration of several aspects of the disclosure. Any equivalent embodiments are intended to be within the scope of this disclosure. Indeed, various modifications of the disclosure in addition to those shown and described herein will become apparent to those skilled in the art from the foregoing description, which do not depart from the spirit or scope of the present inventive discovery. Such modifications are also intended to fall within the scope of the appended claims.

All references, patents or applications, U.S. or foreign, cited in the application are hereby incorporated by reference as if written herein in their entireties. Where any inconsistencies arise, material literally disclosed herein controls.

1. A method of treatment of alcohol use disorder in a subject, comprising administering to the subject an intranasal formulation comprising a therapeutically effective amount of naltrexone or a pharmaceutically acceptable salt thereof.

2. The method as recited in claim 1, wherein the intranasal formulation comprising naltrexone is administered prior to ingestion of alcohol.

3. The method as recited in claim 1, wherein the intranasal formulation comprising naltrexone is administered about 1-2 hours prior to ingestion of alcohol.

4. The method as recited in claim 1, wherein the intranasal formulation comprising naltrexone is administered about 0.5 to about 1 hours prior to ingestion of alcohol.

5. The method as recited in claim 1, wherein the intranasal formulation comprising naltrexone is administered about 10 to about 30 minutes prior to ingestion of alcohol.

6. The method as recited in claim 1, wherein the intranasal formulation comprising naltrexone is administered about 5 to about 10 minutes prior to ingestion of alcohol.

7. The method as recited in claim 1, wherein the intranasal formulation comprising naltrexone is administered contemporaneously with the ingestion of alcohol.

8. The method as recited in claim 1, wherein the intranasal formulation comprising naltrexone is administered within 0.5 hours after commencement of ingestion of alcohol.

9. The method as recited in claim 1, wherein the intranasal formulation comprises an aqueous solution.

10. The method as recited in claim 1, wherein each dose of the formulation comprises about 2 to about 12 mg naltrexone or a salt or hydrate thereof.

11. The method as recited in claim 10, wherein each dose of the formulation comprises about 2 to about 8 mg naltrexone or a salt or hydrate thereof.

12. The method as recited in claim 11, wherein each dose of the formulation comprises about 4 mg naltrexone or a salt or hydrate thereof.

13. The method as recited in claim 1, wherein about 0.05-about 0.2 mL of said formulation is delivered to the subject.

14. The method as recited in claim 13, wherein about 0.1 mL of said formulation is delivered to the subject.

15. The method as recited in claim 13, wherein said formulation is at a concentration of 40 mg/mL.

16. The method as recited in claim 1, wherein the intranasal formulation is administered as a single administration to one nostril.

17. The method as recited in claim 1, wherein the intranasal formulation is administered as two administrations, one to each nostril.

18. The method as recited in claim 1, wherein the intranasal formulation additionally comprises an absorption enhancer.

19. The method as recited in claim 18, wherein the absorption enhancer is selected from the group consisting of benzalkonium chloride, chitosans, cyclodextrins, deoxycholic acid, dodecyl maltoside, glycocholic acid, laureth-9, taurocholic acid, and taurodihydrofusidic acid.

20. The method as recited in claim 18, wherein the absorption enhancer is an alkylsaccharide.

21.-80. (canceled)
